(19) United States
(12) Patent Application Publication
(10) Pub. No.: US 2026/0295217 A1
(43) Pub. Date: Oct. 1, 2026

(54) ARTICULABLE TUBE ASSEMBLIES

(71) Applicant: Fortimedix Assets II B.V., Geleen (NL)

(72) Inventor: Mattheus Hendrik Louis Thissen, Swalmen (NL)

(21) Appl. No.: 19/635,451

(22) Filed: Mar. 31, 2026

Related U.S. Application Data
(60) Provisional application No. 64/022,309, filed on Mar. 30, 2026.
(60) Provisional application No. 64/022,315, filed on Mar. 30, 2026.

(30) Foreign Application Priority Data
Apr. 1, 2025 (NL) 2040096

(51) Int. Cl. A61M 25/01; A61B 17/00; A61M 25/00

(52) U.S. Cl. CPC A61M 25/0138; A61B 2017/00309; A61M 2025/0004

(57) ABSTRACT

According to some embodiments, the present technology includes an elongate member having a proximal portion, a distal portion, an actuation region at the proximal portion, and an articulable region at the distal portion. The elongate member can further comprise a first elongate tube and a second elongate tube coaxial with the first elongate tube. The first elongate tube can comprise a first sidewall and have a first flexible region along which the first elongate tube is configured to bend. The second elongate tube can comprise a second sidewall having a second flexible region comprising a plurality of helical strands formed of the second sidewall. The second flexible region can be at least partially axially aligned with the first flexible region.

### CROSS-REFERENCE TO RELATED APPLICATION(S)

The present application claims the benefit of priority of U.S. Provisional Application No. 64/022,309, filed Mar. 30, 2026, U.S. Provisional Application No. 64/022,315, filed Mar. 30, 2026, and Dutch Patent Application No. 2040096, filed Apr. 1, 2025, each of which is incorporated by reference herein in its entirety.

### TECHNICAL FIELD

The present technology relates to articulable tube assemblies and associated systems and methods of use.

### BACKGROUND

Articulating medical devices, such as steerable catheters, robotic end effectors, diagnostic tools, etc. are widely used. Some are utilized in minimally invasive medical procedures to navigate through tortuous anatomical pathways and reach target locations within the human body. Conventional articulating medical devices typically employ steering wires or pull wires that extend along the length of the devices and are mechanically coupled to the distal tip. When tension is applied to these steering wires through a proximal control mechanism, the distal end of the catheter deflects in a predetermined direction to facilitate navigation through complex vascular or anatomical structures.

However, steering wire-based catheter systems present several technical challenges that can limit their effectiveness and precision during medical procedures. For example, the mechanical coupling between the steering wires and the catheter tip may result in unpredictable deflection characteristics. Additionally, the transmission of force from the proximal control mechanism to the distal tip through the steering wires can be inconsistent due to wire material properties and friction losses along the catheter shaft. These factors can lead to reduced responsiveness, imprecise tip positioning, and difficulty maintaining desired catheter orientations during complex navigational maneuvers, thereby potentially compromising procedural outcomes and requiring additional time and effort from medical practitioners.

Accordingly, there is a need for improved steering devices for medical procedures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Many aspects of the present disclosure can be better understood with reference to the following drawings. The components in the drawings are not necessarily to scale and show different features, examples, or aspects of the present technology. Instead, emphasis is placed on illustrating clearly the principles of the present disclosure.

FIG. 1 depicts an example articulable system, in accordance with several embodiments of the present technology.

FIG. 2A depicts an exploded view of an example steerable tube assembly, in accordance with several embodiments of the present technology.

FIG. 2B depicts a detailed view of a portion of an example steerable tube assembly, in accordance with several embodiments of the present technology.

FIG. 3 shows an example intermediate member in accordance with several embodiments of the present technology.

FIG. 4 depicts an unwrapped view of an example tube with tendons in an example steerable tube assembly, in accordance with several embodiments of the present technology.

FIG. 5 depicts an unwrapped view of an example tube with tendons in an example steerable tube assembly, in accordance with several embodiments of the present technology.

FIG. 6 shows a portion of a tube having a plurality of fracture elements in accordance with several embodiments of the present technology. In FIG. 6, the fracture elements are shown in an unbroken state.

FIG. 7A shows a helical structure in accordance with several embodiments of the present technology. In FIG. 7A, the helical structure is shown in a straight configuration.

FIG. 7B shows the helical structure of FIG. 7A in a bent configuration in accordance with several embodiments of the present technology.

FIGS. 8A-8C schematically depict a plurality of helical strands separated by spacers in accordance with several embodiments of the present technology.

FIG. 9A shows helical and flexible structures of a tube assembly in accordance with several embodiments of the present technology.

FIG. 9B shows helical and flexible structures of a tube assembly in accordance with several embodiments of the present technology.

FIG. 10 shows a helical assembly in accordance with several embodiments of the present technology.

FIG. 11A shows a flexible structure of the prior art.

FIG. 11B shows a flexible structure of the prior art.

FIG. 12A is a perspective view of a flexible structure configured in accordance with several embodiments of the present technology.

FIG. 12B is a side view of the flexible structure of FIG. 12A.

FIG. 12C is a side view of the flexible structure of FIGS. 12A and 12B, shown in a curved configuration in accordance with several embodiments of the present technology.

FIG. 12D is a laid-flat view of the flexible structure of FIGS. 12A-12C in accordance with several embodiments of the present technology.

FIG. 12E is an enlarged view of a portion of the flexible structure shown in FIGS. 12A-12D.

FIG. 12F shows a finite element analysis of a portion of a strut of the flexible structure shown in FIGS. 12A-12E.

FIG. 12G shows a finite element analysis of a portion of a strut of the flexible structure of the prior art, shown in FIG. 11B.

FIGS. 13A and 13B are perspective and side views, respectively, of a flexible structure configured in accordance with several embodiments of the present technology.

FIG. 14 is a perspective view of a flexible structure configured in accordance with several embodiments of the present technology.

FIG. 15 is a perspective view of a flexible structure configured in accordance with several embodiments of the present technology.

FIG. 16A is a perspective view of an elongate member configured in accordance with several embodiments of the present technology.

FIG. 16B is a perspective view of a portion of the elongate member shown in FIG. 16A.

FIG. 17A is a perspective view of an elongate member configured in accordance with several embodiments of the present technology.

FIG. 17B is a perspective view of a portion of the elongate member shown in FIG. 17A.

FIG. 18 shows a rotatable tube assembly in accordance with several embodiments of the present technology.

FIG. 19 is a side view of a distal region of the rotatable tube assembly of FIG. 18, in accordance with several embodiments of the present technology.

FIG. 20 is a side view of a distal region of the rotatable tube assembly of FIG. 18 with the first member removed, in accordance with several embodiments of the present technology.

FIG. 21 is a laid flat view of the rotary actuator of the second elongate tubular member of FIG. 20, in accordance with several embodiments of the present technology.

FIG. 22 is a laid flat view of another example rotary actuator in accordance with several embodiments of the present technology.

FIGS. 23A and 23B depict various slot angles in accordance with several embodiments of the present technology.

FIG. 24 shows the second elongate tubular member of FIG. 18, coupled to an end effector, in accordance with several embodiments of the present technology.

FIG. 25 is an isolated view of the end effector of FIG. 24, shown attached to an elongate control member, in accordance with several embodiments of the present technology.

FIG. 26 shows the end effector as depicted in FIG. 25 but with the clevis removed.

### DETAILED DESCRIPTION

The present technology relates to articulable tube assemblies and associated systems and methods of use. In particular, some embodiments of the present technology relate to various helical structures and tube assemblies incorporating the same. A recurring challenge in laser-cut tube designs arises when small bend radii are required: achieving the requisite flexibility demands removal of substantial material from the laser-cut portion, which in turn compromises the structural integrity of that region and diminishes its capacity to bear axial and torsional loads —loads that are inherently present when steering tendons are used to articulate a distal segment or when end effectors are coupled to the distal end of the instrument. The present technology addresses these competing mechanical demands by providing a tube assembly comprising coaxial tubes having helical cut patterns of opposite chirality. The opposing helical geometry allows the assembly to achieve small bend radii while simultaneously maintaining the capacity to withstand high axial and torsional loads. In some embodiments, one or both of the helically cut tubes are configured to avoid path length differences amongst the helical strands when bending, thereby providing the tube with a uniform diameter when bending, even around relatively sharp turns. The helical tube structures of the present technology can be aligned with one or more flexible regions of a steerable tube assembly, such as an articulable region and/or passively bendable region, thereby providing structural reinforcement to flexible regions at other layers of the instrument.

Some embodiments of the present technology relate to laser-cut patterns to be employed along a flexible region of an elongate tubular member. For example, elongate tubular members used in steerable medical instruments often incorporate laser-cut patterns along flexible regions to enable controlled bending and articulation (whether passive or actively controlled with steering members). However, conventional laser-cut geometries may exhibit non-uniform mechanical behavior, providing inconsistent axial stiffness or bending resistance depending on the direction of applied force. The present technology addresses these limitations by providing laser-cut patterns having unique geometries configured to permit omnidirectional bending while maintaining uniform axial stiffness throughout the flexible region. Such laser-cut patterns may be employed along a steerable region of the tubular member or at any other location along its length where controlled flexibility is desired, offering versatility in instrument design across a range of clinical applications.

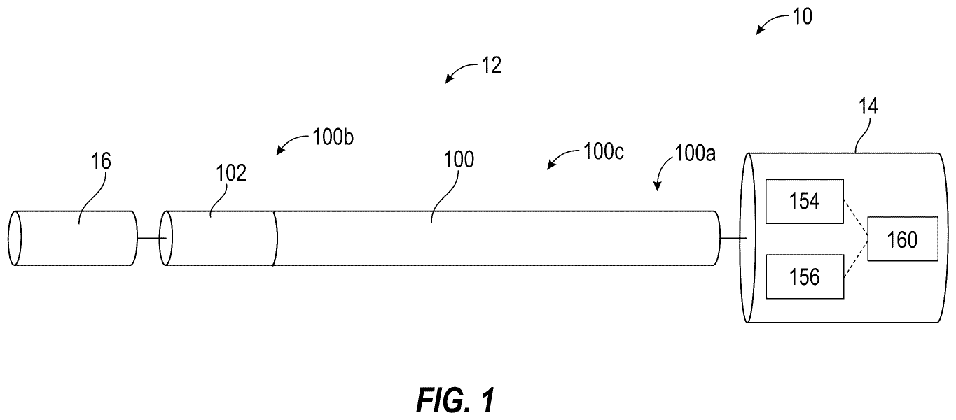

Specific details of several embodiments of the technology are described below with reference to FIGS. 1-26. As used herein, the terms “proximal” and “distal” are defined with respect to an operator, e.g., a robot or physician that operates the instrument, catheter, or endoscope. For example, a proximal portion should be construed as a portion located closer to the robot or physician and a distal portion should be construed as a portion located farther from the robot or physician.

### I. Articulable Tube Assemblies

The present technology relates to a tube for an articulable device such as a medical instrument or catheter (e.g., for non-invasive procedures, such as endoscopic, laparoscopic, etc. and/or invasive types of applications, such as in surgery) or other articulable devices. Articulable tube assemblies according to the present technology can be used in in a variety of medical applications, including but not limited to accessing and/or navigating the vasculature (arteries and/or veins), the heart (e.g., the chambers of the heart), the respiratory system (e.g., the mouth, nose, trachea, bronchi, bronchioles, etc.) and/or the gastrointestinal system (e.g., the esophagus, small intestine, the colon, etc.). The articulable tube assemblies disclosed herein can be used in both medical and non-medical applications. Examples of the latter include inspection and/or repair of mechanical and/or electronic hardware at locations that are difficult to reach (e.g., optical devices, plumbing devices, etc.). Hence, terms used in the following description such as endoscopic application or invasive instrument must be interpreted in a broad manner.

In some embodiments, an articulable device may include an articulable tube assembly having one or more articulable regions along its length, where the one or more articulable regions are configured to articulate (e.g., bend, curve, etc.). In some embodiments, an articulable region may be configured to articulate passively (e.g., in response to articulating movement of a stylet or an outer sheath telescopically engaged with the articulable region, in response to an anatomical environment, etc.). Additionally or alternatively, in some embodiments, an articulable region may be configured to articulate in response to one or more steering inputs. For example, in some embodiments, the articulable tube assembly may be configured to receive one or more steering inputs at a first location along the length of the articulable tube assembly, and the articulable tube assembly may be configured to communicate and/or transform the steering input(s) into articulation at one or more articulable regions located at at least a second location along the length of the articulable tube assembly, where the second location is distal to the first location. In some embodiments, the one or more steering inputs may be located at a proximal portion of the articulable tube assembly, and the articulable region(s) may be located at an intermediate portion and/or distal portion of the articulable tube assembly.

In some embodiments, the articulable tube assembly may include multiple elongate members (e.g., tubular members) that are arranged coaxially, such as in a nested manner. The multiple elongate members may have respective articulable regions that are longitudinally aligned, such that when the multiple elongate members are assembled together in an articulable tube assembly, their articulable regions are configured to be shaped and articulate in tandem. In some embodiments, an actuating input may be applied to a portion (e.g., proximal portion, intermediate portion, or other portion that is longitudinally distanced from an articulable region) of one elongate member (e.g., an outer elongate member), and that actuating input may be communicated to a steering feature such as a tendon (as described in further detail below) on an underlying elongate member to cause collective articulation of the multiple assembled elongate members. “Steerable region” is used herein to refer to both passively bendable and/or flexible regions of the articulable tube assembly and/or the elongate members, and to steerable regions configured to be deflected in a desired direction by a desired amount in response to a steering input from a user. Some flexible regions may be both passively bendable and steerable, some flexible regions may be only passively bendable (i.e., not controlled by any steering tendons), and some flexible regions may bend only in response to a steering input. The actuating input may be generated by a manually operated or motor-operated device, or combination of both. In some instances the motorized actuating device may be robotically controlled operating either automatically or under human control. In some embodiments, the control interface 14 includes a robotic steering interface. In some embodiments, the control interface 14 comprises a handheld motorized device.

Further details of example articulable devices and articulable tube assemblies are described below. Although examples of an articulable tube assembly are primarily described herein as a steerable tube assembly, it should be understood that at least some aspects of an articulable tube assembly may be similarly incorporated in a tube assembly that is passively articulated (e.g., in response to articulating movement of a stylet arranged within the tube assembly, movement of an outer tube arranged outside the tube assembly, in response to contact with the anatomy and/or another structure, and/or the like).

FIG. 1 is a schematic illustration of an example articulable system 10 including a steerable device 12 and a control interface 14 configured to be operably coupled to the steerable device 12 to at least partially control operation of the steerable device 12. The control interface 14 can be fully manual (e.g., a handle), fully motorized (e.g., robotic), or a combination of manual and motorized. As shown schematically in FIG. 1, the steerable device 12 may include a steerable tube assembly 100 and at least one end effector 16 configured to be coupled to the steerable tube assembly 100. In some embodiments, the articulable system 10 does not include the end effector 16.

The tube assembly 100 includes a distal portion 100b configured to be positioned in a body lumen, a proximal portion 100a configured to be coupled to the control interface 14, and an intermediate portion 100c extending therebetween. The tube assembly 100 includes a steerable region 102 at the distal portion 100b that is configured to be selectively deflected in response to a steering input at the proximal portion 100a, as detailed herein. The intermediate portion 100c may be passively bendable and/or flexible (e.g., to accommodate one or more turns in the body lumen) but not steerable. In some embodiments, the intermediate portion is not bendable. The steerable region 102 may be configured to deflect in a single plane (e.g., bi-directional) or multiple planes (e.g., multi-dimensional). While only a single steerable region 102 is shown in FIG. 1, in some examples the tube assembly 100 may have two or more steerable regions 102, each independently controllable at the proximal portion 100a. In some embodiments, the tube assembly 100 does not include a steerable region 102 and instead comprises a tube configured to transition between flexible and rigid configurations so as to lock the tube in a desired shape. As used herein, “medical instrument” can refer to the articulable system, the steerable device, and/or the tube assembly, whether steerable or not.

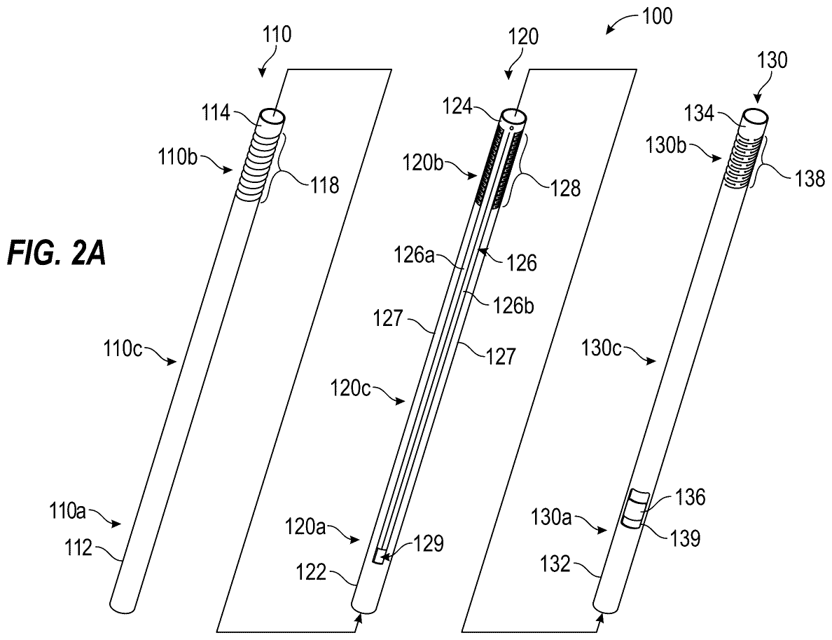

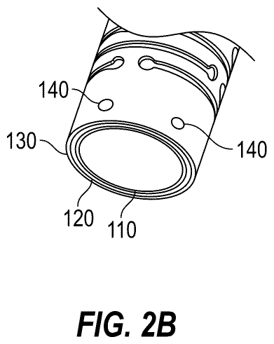

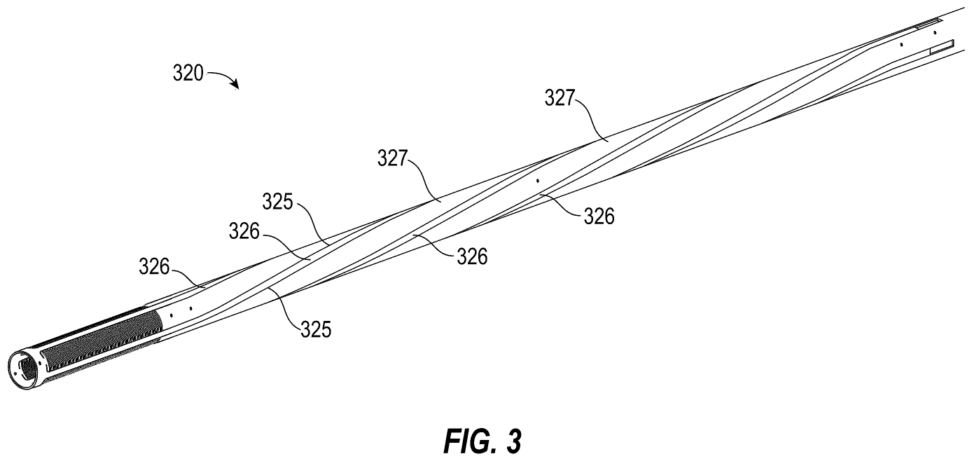

In some embodiments, the control interface 14 can include one or more drivers 154, one or more sensors 156, and a controller 160 operatively coupled to the driver(s) 154 and sensor(s) 156. The driver(s) 154 can be operatively coupled to the proximal end portions of one or more steering tendons 126 (discussed below with reference to FIG. 2A) of the tube assembly 100 to push/pull the steering tendons 126 to cause articulation of the steerable region 102. In some embodiments, each of the steering tendons 126 is operatively coupled to a different, independently controlled driver 154. In other embodiments, some or all of the steering tendons 126 may be operatively coupled to the same driver 154. The sensor(s) 156 can be configured to measure and/or detect a position and/or change in position (e.g., displacement), such as a longitudinal position of the proximal end portions of the steering tendon(s) 126 (discussed below with reference to FIG. 2A). In some cases, movement of the steering tendon 126 at the proximal portion 100a may be along a linear path (as shown in FIGS. 2A and 3), regardless of whether the steering tendon 126 takes a linear or helical path along the intermediate portion 100c of the tube assembly 100. In other cases, movement of the steering tendon 126 at the proximal portion 100a may be along a helical path (as shown in FIGS. 2A and 3), regardless of whether the steering tendon 126 takes a linear or helical path along the intermediate portion 100c of the tube assembly 100a. In either case, the sensor(s) 156 is configured to measure a change in position, which may be referred to as an “axial” or “longitudinal” position herein. The controller 160 can include one or more processors and memory having instructions configured to be executed by the one or more processors. The driver(s) 154 and/or sensor(s) 156 are configured to transmit displacement data to the controller 160, which the controller 160 may process to control various operations of the tube assembly 100 and/or steerable device 12 as well as provide information to the user, as discussed in greater detail below.

The sensor(s) 156 may be any suitable sensor for monitoring movement or displacement. For example, the sensor(s) 156 may be a non-contact linear displacement sensor, such as an optical encoder (e.g., utilizing either incremental or absolute encoding schemes), a magnetic encoder (e.g., utilizing magnetic fields and Hall effect sensors or magnetoresistive elements to detect position), a linear variable differential transformer (e.g., configured to measure displacement through electromagnetic coupling, generating an analog voltage proportional to the position of a movable core within transformer coils), a capacitive displacement sensor (e.g., configured to measure change in capacitance between parallel plates as their separation varies), a potentiometric sensor (e.g., uses a resistive element and sliding contact to generate a voltage proportional to linear position), laser displacement sensor and time-of-flight systems (e.g., configured to provide non-contact measurement by analyzing reflected light), and/or others. As such, the sensors 156 are not physically coupled to the tendons. In other embodiments, one, some, or all of the tendons may be physically coupled to a linear displacement sensor.

The driver(s) 154 utilized with the present technology may include one or more motors and one or more sensors configured to provide information to the controller 160 for analysis and/or operating one or more components of the system. The motors can be operatively coupled to the steering tendons 126 via one or more connectors such that movement of the motor causes movement of the attached steering tendon 126. The sensor(s) can include sensors configured to measure current supplied to the motor (which can be used to measure torque), dedicated force sensors (e.g., load cells, fiberoptic force sensors, capacitive force sensors, etc.), and/or position sensors such as motor-side encoders (e.g., incremental encoders, absolute encoders, hall effect sensors, optical encoders, magnetic encoders, etc.) and/or direct linear position sensors. In some cases, the drive mechanism comprises a leadscrew mechanism which converts rotational motion of the motor to axial movement of the associated steering tendon 126. In such cases, the position of the steering tendon 126 can be determined based on the motor rotations. The data obtained by the sensors (e.g., motor current, motor position or change in position, force, etc.) can be used by the controller 160 to determine one or more operational parameters of the system, such as position or change of position in a steering tendon 126 and/or amount of axial force exerted on the steering tendon by the associated driver 154.

In some embodiments, the control interface 14 does not include one or more drivers 154 and instead the steering tendons 126 are controlled manually by a user. In such embodiments, the control interface 14 may comprise a handle having one or more actuators that are operatively coupled to the steering tendons 126 to control movement of the steering tendons 126. Such actuators amy comprise one or more sliders, knobs, buttons, and other manual controls. The handle may further include the sensor(s) 156 for detecting a position or change in position of the steering tendons 126. The controller 160 may be integrated into the handle, separate from the handle and configured to be in communication with the sensors of the handle, or distributed across the handle and one or more separate components.

As shown schematically in FIG. 1, the steerable device 12 and/or tube assembly 100 may be coupled at an end portion (e.g., a distal end portion) to the at least one end effector 16. The end effector 16 may include a tool (e.g., graspers, scissors, ablation tool, etc.), a sensor (e.g., camera, electrode, etc.) and/or any suitable instrument. Components useful for operation of the end effector 16, such as wires for actuation of a tool, signal transmission, and/or electrical power transmission, may be passed along one or more lumens defined within the tube assembly 100. In some embodiments, no end effector 16 is coupled to the steerable device 12 and/or tube assembly 100. For example, the steerable device 12 may be a catheter (e.g., delivery catheter). In some examples, the end effector 16 may be a separate instrument comprising an elongate delivery member coupled to a tool (e.g., graspers, scissors, ablation tool, etc.), a sensor (e.g., camera, electrode, etc.) at its distal end. The instrument may be configured to be delivered through a tube assembly of the present technology.

As described above, in some embodiments, a tube assembly 100 may include multiple, coaxial elongate tubular members. For example, as shown in FIG. 2A, an example tube assembly 100 may include an inner member 110, an outer member 130, and an intermediate member 120 arranged between the inner member 110 and the outer member 130. The inner member 110 may be positioned within a lumen of the intermediate member 120, and the intermediate member 120 may be positioned with a lumen of the outer member 130. Although FIG. 2A illustrates an example in which the tube assembly 100 includes a single intermediate member 120, it should be understood that in some embodiments, the tube assembly 100 may include two, three, or more intermediate members arranged between the inner member 110 and the outer member 130, where each intermediate member may have a respective set of operable features. Moreover, a tube assembly 100 may include any suitable number of nested, coaxial tubular members (e.g., two, three, four, or more than four); however, for the sake of simplicity of explanation, a representative tube assembly 100 with three elongate members is primarily described herein. In some embodiments, an articulable device may include only a single tubular member incorporating at least some features of the elongate members as described herein.

The inner member 110 may include a proximal portion 110a including a proximal end portion 112, a distal portion 110b including a distal end portion 124, and an intermediate portion 110c extending between the proximal and distal portions 110a, 110b of the inner member 110. The proximal portion 110a may be aligned with the proximal portion 100a of the tube assembly 100, the intermediate portion 110c may be aligned with the intermediate portion 100c of the tube assembly 100, and the distal portion 110b may be aligned with the distal portion 100b of the tube assembly 100. The distal portion 110b of the inner member 110 may include at least one flexible region 118 configured to articulate (e.g., bend, curve, flex, etc.) and that is axially aligned with the steerable region 102 when the tube assembly 100 is assembled. The distal end portion 114 of the inner member 110, which is distal of the flexible region 118, may comprise a rigid (e.g., non-articulable) band. Although FIG. 2A illustrates an example in which the inner member 110 includes one flexible region 118 located generally in the distal portion 110b, it should be understood that in some embodiments, the inner member 110 may include two or more (e.g., two, three, four, more than four, etc.) flexible regions 118 arranged along the distal portion 110b. In some embodiments, adjacent flexible regions 118 can be separated by a rigid portion and/or band.

The intermediate portion 110c of the inner member 110 may be passively bendable and/or flexible (e.g., to accommodate one or more turns in the body lumen) but not steerable. In some embodiments, the intermediate portion 110c may be rigid and thus not bendable (e.g., maintains a linear configuration).

The flexible region 118 may include one or more articulating features configured to enable the flexible region 118 to assume a suitable articulated shape (e.g., bent, curved, etc.). For example, as shown in FIG. 2A, the flexible region 118 may include a plurality of cuts and/or slits, such as circumferentially extending cuts and/or helically extending cuts, etc., configured to improve the flexibility and/or bendability of the respective elongate member along that region. As described in further detail herein, the cuts may, for example, be formed by cutting any suitable pattern from the wall of the inner member 110. In some embodiments, dimensional aspects of the cut pattern in the flexible region 118 (e.g., longitudinal length of the flexible region 118, size and shape of cuts, spacing between cuts, etc.) may be selected to accommodate specific performance requirements, such as with regard to bending angle, bending flexibility, longitudinal stiffness, and/or radial stiffness of the flexible region 118.

The intermediate portion 110c of the inner member 110 may also include one or more articulating features, similar to the articulating features described above, that enables the intermediate portion 110c to passively bend and flex.

Similar to the inner member 110, the outer member 130 may include a proximal portion 130a including a proximal end portion 132, a distal portion 130b including a distal end portion 134, and an intermediate portion 130c extending between the proximal and distal portions 130a, 130b of the outer member 130. The proximal portion 130a may be aligned with the proximal portion 100a of the tube assembly 100, the intermediate portion 130c may be aligned with the intermediate portion 100c of the tube assembly 100, and the distal portion 130b may be aligned with the distal portion 100b of the tube assembly 100. The distal portion 130b of the outer member 130 may include at least one flexible region 138 configured to articulate (e.g., bend, curve, flex, etc.) and that is axially aligned with the steerable region 102 when the tube assembly 100 is assembled. The distal end portion 134 of the outer member 130, which is distal of the flexible region 138, may comprise a rigid (e.g., non-articulable) band. Although FIG. 2A illustrates an example in which the outer member 130 includes one flexible region 138 located generally in the distal portion 130b, it should be understood that in some embodiments, the outer member 130 may include two or more (e.g., two, three, four, more than four, etc.) flexible regions 138 arranged along the distal portion 130b. In some embodiments, adjacent flexible regions 138 can be separated by a rigid portion and/or band.

The one or more flexible regions 138 of the outer member 130 may be constructed in a similar manner to the flexible region(s) 118 of the inner member 110. The flexible regions 118 and 138 may have the same or different cut configurations and/or cut patterns.

The intermediate portion 130c of the outer member 130 may be passively bendable and/or flexible (e.g., to accommodate one or more turns in the body lumen) but not steerable. The intermediate portion 130c may also include one or more articulating features, similar to the articulating features described above, that enables the intermediate portion 130c to passively bend and flex. In some embodiments, the intermediate portion 130c may be rigid and thus not bendable (e.g., maintains a linear configuration).

The intermediate member 120 may include a proximal portion 120a including a proximal end portion 122, a distal portion 120b including a distal end portion 124, and an intermediate portion 120c extending between the proximal and distal portions 120a, 120b of the intermediate member 120. The proximal portion 120a may be aligned with the proximal portion 100a of the tube assembly 100, the intermediate portion 120c may be aligned with the intermediate portion 100c of the tube assembly 100, and the distal portion 120b may be aligned with the distal portion 100b of the tube assembly 100. The distal portion 120b of the intermediate member 120 may include at least one flexible region 128 configured to articulate (e.g., bend, curve, flex, etc.) and that is axially aligned with the steerable region 102 when the tube assembly 100 is assembled. The distal end portion 124 of the intermediate member 120, which is distal of the flexible region 128, may comprise a rigid (e.g., non-articulable) band. Although FIG. 2A illustrates an example in which the intermediate member 120 includes one flexible region 128 located generally in the distal portion 120b, it should be understood that in some embodiments, the intermediate member 120 may include two or more (e.g., two, three, four, more than four, etc.) flexible regions 128 arranged along the distal portion 120b. In some embodiments, adjacent flexible regions 128 can be separated by a rigid portion and/or band.

The one or more flexible regions 128 of the intermediate member 120 may be constructed in a similar manner to the flexible region(s) 118 of the inner member 110. The flexible regions 118, 128, and 138 may have the same and/or different cut configurations and/or cut patterns.

The intermediate portion 120c of the intermediate member 120 may be passively bendable and/or flexible (e.g., to accommodate one or more turns in the body lumen) but not steerable. The intermediate portion 120c may also include one or more articulating features, similar to the articulating features described above, that enables the intermediate portion 120c to passively bend and flex. In some embodiments, the intermediate portion 120c may be rigid and thus not bendable (e.g., maintains a linear configuration).

The inner member 110, the intermediate member 120, and the outer member 130 may be assembled to form a combined unit that defines the tube assembly 100. For example, the inner member 110 may be inserted into the intermediate member 120, and the combined inner member 110 and intermediate member 120 subassembly may be inserted into the outer member 130, although any order of insertion may be possible. In some embodiments, only two elongate members (e.g., the inner member 110 and the intermediate member 120, the inner member 110 and the outer member 130, or the intermediate member 120 and the outer member 130) may be assembled to form a combined unit within the tube assembly 100. In some embodiments, the distal end portions 114, 124, 134 of the inner member 110, the intermediate member 120, and/or the outer member 130, respectively, may be coupled to one another so as to be fixed together. In some examples, the proximal end portions 112, 122, 132 of the inner member 110, the intermediate member 120, and/or the outer member 130, respectively, may be coupled to one another so as to be fixed together. Moreover, the inner member 110, intermediate member 120, and outer member 130 may be fixed to one another at other locations along their respective lengths. As but one example, the inner member 110 may be inserted into the lumen of the intermediate member 120, and one or more spots welds may be applied at various locations along their respective distal, intermediate, and proximal portions. The inner-intermediate member assembly can be inserted into the lumen of the outer member 130, and additional spot welds may be applied at various locations along their respective distal, intermediate, and proximal portions. In those embodiments in which the tube assembly 100 includes two steerable regions (and thus each of the inner member 110, intermediate member 120, and outer member 130 have two flexible regions, each of which is longitudinally aligned with the corresponding flexible region in the radially adjacent tube), the inner member 110, the intermediate member 120, and the outer member 130 may be fixed to one another at the respective rigid bands on either side of each flexible region.

Such coupling of the inner member 110, the intermediate member 120, and/or the outer member 130 may be accomplished in any suitable manner, such as with epoxy, welding (e.g., laser welding), or mechanical interfit (e.g., press fit). For example, FIG. 2B provides a more detailed view of a distal end portion of an example tube assembly including three co-axially arranged layers or tubular members, including inner member 110, intermediate member 120, and outer member 130. The distal end portions 114, 124, 134 of the inner member 110, the intermediate member 120, and the outer member 130 may be fixedly attached to one another, such as with one or more spot welds 140. In some embodiments, multiple spot welds 140 may be arranged circumferentially around the tube assembly, either equally distributed or unequally distributed around the circumference of the tube assembly 100. The spot welds 140 may be arranged at the proximal end portions 112, 122, and/or 132, and/or at the distal end portions 114, 124, and/or 134, and/or at any suitable axial location along the length of the tube assembly 100.

As shown in FIG. 2A, the intermediate member 120 may further include one or more steering tendons 126, each associated with and/or extending through a respective flexible region 128, such that movement of a tendon 126 causes articulation and/or deflection of its corresponding flexible region 128. In some embodiments, a distal end portion of a steering tendon 126 may be coupled to (e.g., integrally formed with, or attached such as via at least one weld) a rigid portion and/or band just distal of the corresponding flexible region 128. For example, in FIG. 2A, the distal end portion of steering tendon 126 is integral with the rigid band comprising the distal end portion 124 of the intermediate member 120. A proximal end portion of the steering tendon 126 is operatively coupled to the driver 154 that pushes/pulls the proximal end portion of the steering tendon 126 to cause articulation of the steerable region 102.

In some embodiments, a distal end portion of a steering tendon 126 may be coupled to (e.g., integrally formed with, or attached such as via at least one weld) a rigid portion and/or band just distal of the corresponding flexible region 128. For example, in FIG. 2A, the distal end portion of steering tendon 126 is integral with the rigid band comprising the distal end portion 124 of the intermediate member 120. By “integral with” or “integrally formed with” it is meant that the steering tendon 126 and rigid band are cut from the same tube. A proximal end portion of the steering tendon 126 is operatively coupled to the driver 154 that pushes/pulls on proximal end portion of the steering tendon 126 to cause articulation of the steerable region 102.

As shown in FIG. 2A, a tendon 126 may be formed as a longitudinal member or strip extending longitudinally along at least a portion of a wall of the intermediate member 120, for at least a portion of the length of the intermediate member 120. A tendon 126 may be cut from the sidewall of the intermediate member 120 and thus integral with the sidewall of the intermediate member 120. A tendon 126 may be configured to move generally in a longitudinal direction within a respective slot 129 defined by the intermediate member 120 (e.g., cut from the wall of the intermediate member 120). On either side of a tendon 126 or slot 129 can be a tendon-adjacent portion 127 of the sidewall that is not configured to move longitudinally and/or be actuated and is separated from the tendon 126 by a slit. The slit may, for example, be formed by removal of material such as laser cutting, where the width of the slit corresponds to the width of the laser beam. In the example shown in FIG. 2A, the slot 129 is generally linear and extends in a longitudinal direction. However, in some embodiments the slot 129 may be any suitable shape generally extending in a longitudinal direction, such as a helical shape that wraps in a spiral manner around the wall of the intermediate member 120.

Referring still to FIG. 2A, in some examples, one, some, or all of the tendons 126 may be split into multiple sub-strips 126a, 126b between their respective proximal and distal end portions. While the tendon 126 visible in FIG. 2A is split into two sub-strips 126a, 126b, in some embodiments one, some, or all of the tendons 126 may have more sub-strips (e.g., three sub-strips, four sub-strips, five sub-strips, six sub-strips, etc.), and/or one, some, or all of the tendons do not have a split configuration. The tendons 326 in FIG. 3, for example, have a non-split configuration. In some implementations, at least one of the tendons 126 is split (e.g., into any number of sub-strips) while at least another one of the tendons 126 is not split (e.g., is solid along its length). In any of the foregoing embodiments, the sub-strips may terminate at any location along the length of the intermediate member 120, including along the distal end portion 124, flexible region 128, distal portion 120b, intermediate portion 120c, or proximal portion 120a.

In those embodiments in which one, some, or all of the tendons 126 are split into sub-strips, the branched tendons may be coupled to a rope equalizer and/or travel balancer, for example as detailed in U.S. Patent Publication No. 2025/0176801, filed Jan. 31, 2025, which is incorporated by reference herein in its entirety.

A tendon 126 may have any suitable corresponding shape to travel within the corresponding slot 129 (e.g., a helical tendon configured to travel generally in a proximal and/or distal direction within a helical slot). For example, FIG. 3 illustrates an example intermediate member 320 (an example of intermediate member 120) that includes multiple helical tendons 326 that have been obtained after making longitudinal cuts 325 (only a few labeled) in the wall of the intermediate member 320. As shown in FIG. 3, the tendons 326 are, at least in part, spiraling about a longitudinal axis of the intermediate member 320 such that a proximal portion of a given tendon 326 is arranged at a different angular orientation about the longitudinal axis than a distal portion of the same given tendon 326. The tendon-adjacent portions 327 of the sidewall may also extend helically and/or spiraling. In some embodiments, the spiral construction may be such that the proximal end portion of a tendon 326 is arranged at an angularly shifted orientation of 180 degrees about the longitudinal axis relative to the distal end portion of the same tendon 326. However, the angularly shifted orientation between the two end portions of a tendon 326 may be any suitable angle (e.g., 45 degrees, 60 degrees, 75 degrees, 90 degrees, etc.). The spiraling compensates path length changes in the tendons 326 caused by any bending of the intermediate portion of the intermediate member 320, for instance when the tube assembly is inserted in a tortuous path.

In some embodiments, a tendon 126 may be actuated via an actuating input applied to a feature of the outer member 130. For example, as shown in FIG. 2A, in some embodiments, the outer member 130 may include at least one slider 136 configured to move within a respective slot 139 defined by the outer member 130. Each slider 136 may be coupled to an underlying tendon 126 (e.g., via epoxy, spot welding, etc.) such that movement of the slider 136 results in movement of its associated tendon 126. The outer member 130 may include multiple sliders 136, where each slider 136 is coupled to a respective tendon 126, such as selective actuation of the sliders 136 results in selective movement of tendons 126 and thus selective articulation of the articulable regions of the members of the tube assembly. Although the slot 139 is shown in FIG. 2A as generally linear such that the slider 136 moves longitudinally along the outer member 130 (e.g., to push and/or pull a tendon 126 to which the slider 136 is coupled), it should be understood that in some embodiments, the slider 136 may move at least partially in a rotational manner in an arcuate slot 139 (e.g., the slider 136 may be configured to move in a helical manner along a helical slot 139, which may, for example, thereby cause movement of an underlying tendon 126 to which the slider 136 is connected).

Furthermore, although only one tendon 126 is visible in the example shown in FIG. 2A, the intermediate member 120 may include any suitable number of tendons 126. For example, in some embodiments a second tendon 126 may be located on a circumferentially opposite side of the intermediate member 120. For example, two tendons 126 may be circumferentially offset about 180 degrees from one another and operate in an antagonistic manner to articulate an articulable region 128 to which the two tendons 126 are coupled. For instance, a first tendon 126 may be pulled proximally in a longitudinal direction (and/or a second tendon 126 arranged circumferentially opposite to the first tendon 126 may be pushed distally in a longitudinal direction) to cause bending of the articulable region 128 in a first direction. The second tendon 126 may be pulled proximally in a longitudinal direction (and/or the first tendon 126 may be pushed distally in a longitudinal direction) to cause bending of the articulable region 128 in a second direction (e.g., opposite the first direction).

In some embodiments, the intermediate member 120 may include three or more tendons 126 coupled to a common articulable region 128. In some embodiments, multiple tendons 126 may be circumferentially spaced apart in an equidistant manner (e.g., the tendons may be located at equidistant locations as viewed in the tangential direction of the intermediate member 120), though in some embodiments an intermediate member 120 may additionally or alternatively include multiple tendons 126 that are circumferentially spaced apart in an unequal manner.

Additionally or alternatively, the intermediate member 120 may include one or more tendons 126 each coupled to a different flexible region 128. For example, a first pair of antagonistic tendons 126 may be coupled to a first flexible region 128 (e.g., arranged at a first axial location along the length of the intermediate member 120), and a second pair of antagonistic tendons 126 may be coupled to a second flexible region 128 (e.g., arranged, at a second axial location along the length of the intermediate member 120). In some embodiments, the intermediate member 120 may include multiple tendons 126 of substantially equal length, though in some embodiments the intermediate member 120 may additionally or alternatively include multiple tendons 126 of different lengths.

In some embodiments, flexible region(s) of the inner member 110, the intermediate member 120, and/or the outer member 130 may be longitudinally aligned with or at least overlap with one another when the inner, intermediate, and outer members are assembled together in the tube assembly 100. As such, when the flexible region(s) 128 are articulated (via actuation of one or more tendons 126), the underlying flexible region(s) 118 of the inner member 110 and the overlying flexible region(s) 128 of the outer member 130 are passively articulated to follow or generally match the shape of the flexible region(s) 128 of the intermediate member 120.

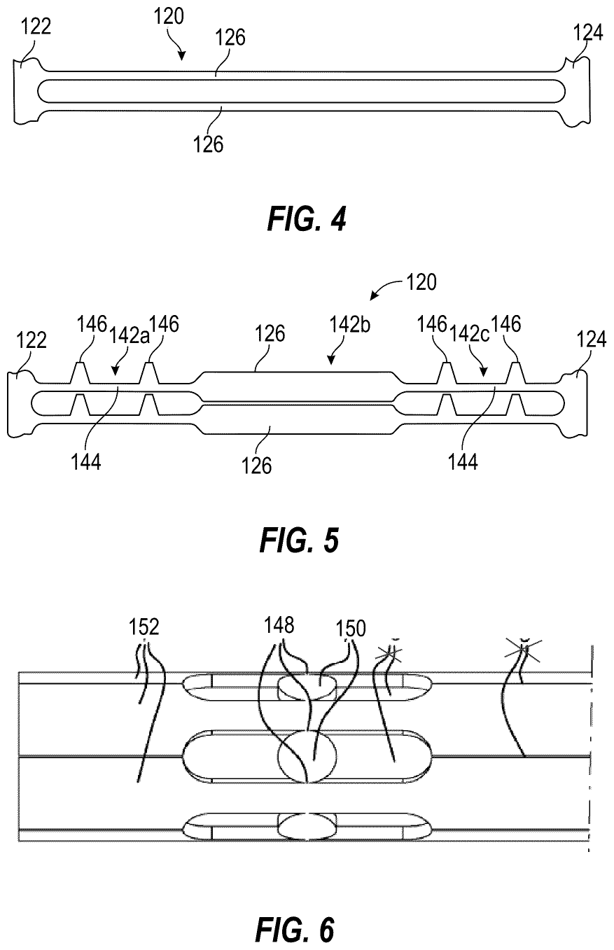

In some embodiments, the intermediate member 120 may include one or more tendons 126 having a substantially uniform cross-section (e.g., width) along its length. For example, FIG. 4 illustrates an example of an intermediate member 120 in an unrolled condition including two parallel tendons 126 having substantially uniform cross-sections along their lengths between the proximal end portion 122 of the intermediate member and the distal end portion 124 of the intermediate member. In the example shown in FIG. 4, the tendons 126 are attached at both the proximal end portion 122 and the distal end portion 124; however, in some embodiments such as that shown in FIG. 2A, the tendons 126 may be attached to only one of the proximal end portion 122 and the distal end portion 124 (e.g., only the distal end portion 124, such as to allow for articulation of an articulable region 128 near the distal end portion 124). The tendons 126 are shown as equally spaced apart, though as described above, in some embodiments, the tendons 126 may be unequally spaced apart. Additionally or alternatively, although the intermediate member 120 shown in FIG. 4 includes two tendons 126, in some embodiments the intermediate member 120 may include three, four, five, six, seven, eight, or more than eight tendons 126.

In some embodiments, one or more tendons 126 may have a varying cross-section (e.g., width) along their lengths. For example, a tendon 126 may have a wider width (e.g., as measured circumferentially in arc length around the intermediate member) at one longitudinal location, compared to its width at another longitudinal location. A wider portion of the tendon 126 may, in some embodiments, function as a spacer between adjacent tendons 126, to help prevent adjacent tendons 126 from buckling in a tangential (e.g., circumferential) direction (e.g., when pushed). However, the intermediate member 120 may include one or more spacers that are implemented in any suitable manner.

For example, FIG. 5 illustrates an example portion of an intermediate member 120 including two adjacent tendons 126 in an unrolled condition. Each tendon 126 includes a first segment 142a, a second segment 142b, and a third segment 142c that are arranged end-to-end along the tendon 126. In the second segment 142b, adjacent tendons 126 are nearly touching each other in the tangential direction such that only a narrow slot is present between the adjacent tendons 126 with a width just sufficient to allow independent movement of each tendon 126. The narrow slot may, for example, be formed by removal of material such as laser cutting, where the width of the slot corresponds to the width of the laser beam.

In the first segment 142a and the third segment 142c, each tendon 126 includes a flexible portion 144 and one or more spacers 146. The flexible portion 144 has a narrower width than the second segment 142b and narrower width than the spacer(s) 146, such that there is a wider gap between adjacent tendons 126. The one or more spacers 146 extend in a tangential direction and may be almost completely bridging the gap between adjacent flexible portions 144 in adjacent tendons 126. The spacer(s) 146 may function to suppress the tendency of the tendons 126 to shift in a tangential direction, thus improving control of the tendons 126 in a tangential direction during actuation of the tendons 126, thereby leading to more control in articulating the articulable region(s) of the tube assembly. The exact shape of the spacer(s) 146 may vary. For example, as shown in FIG. 5 the spacers 146 may have a generally triangular shape, but may alternatively have any suitable shape (e.g., rectangular, semi-circular, etc.). In some embodiments, the spacers 146 may be integrally formed with one or more tendons 126. In some embodiments, the spacers comprise the tendon-adjacent portions of the sidewall.

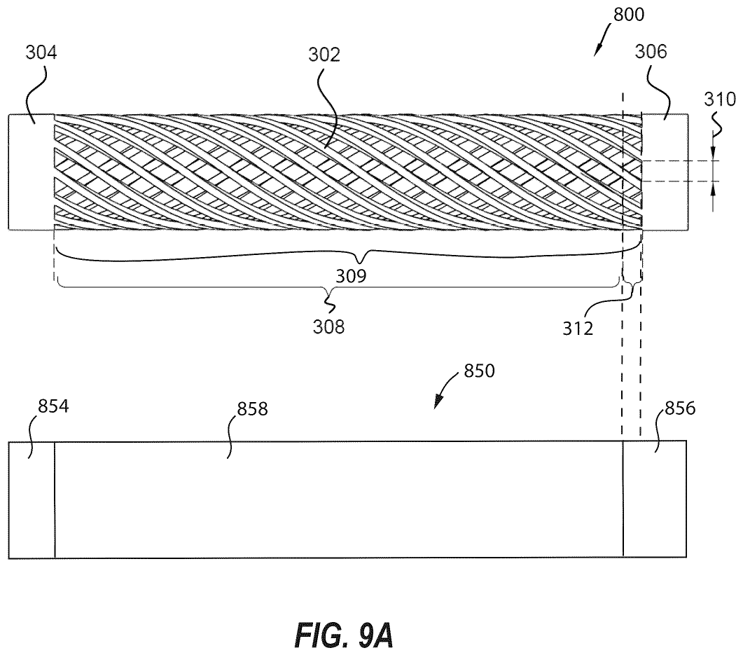

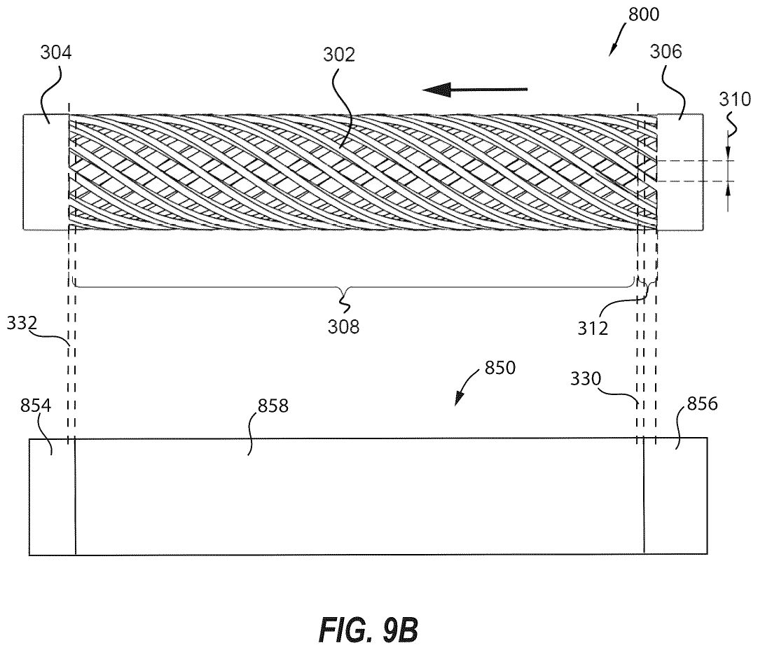

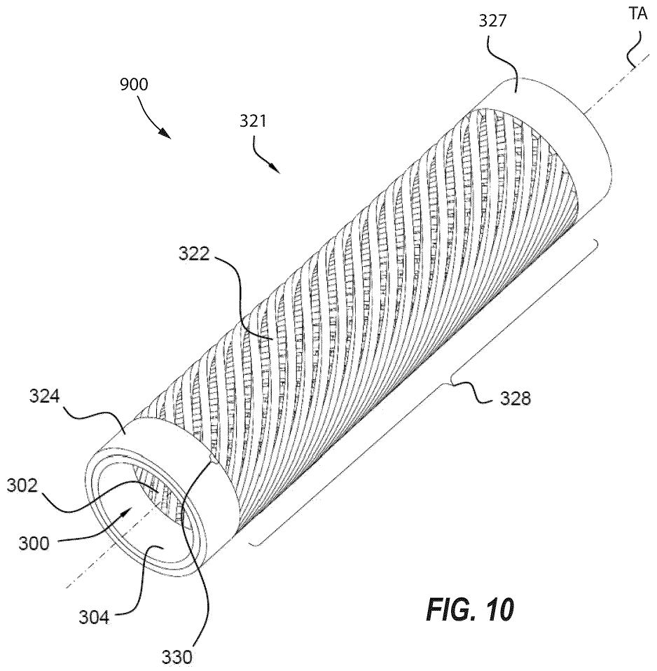

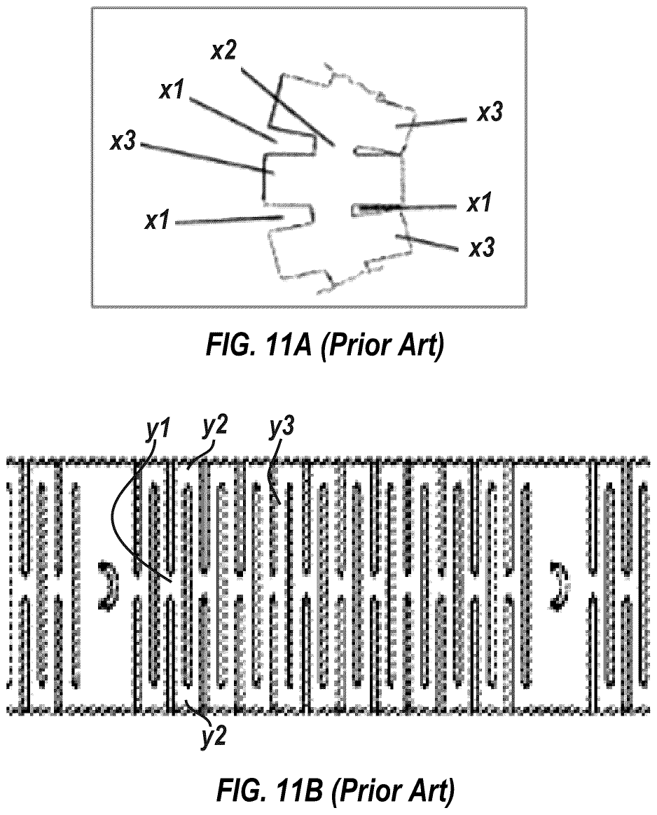

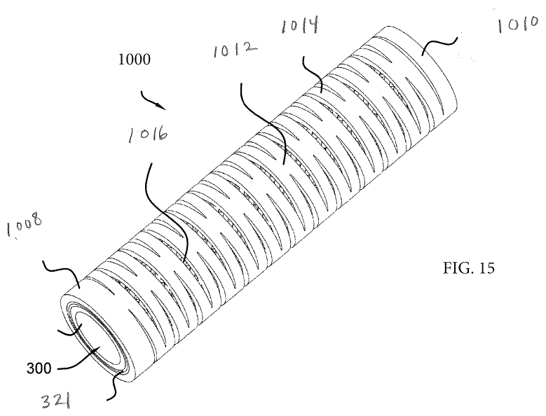

The intermediate member 120 may include any suitable spacers and/or other features, such as those described in FIGS. 4-16 of International Patent Application Publication No. WO2009/112060, FIGS. 6-10B of International Patent Application Publication No. WO2017/082720, FIGS. 15-17 of International Patent Application Publication No. WO2018/067004, FIGS. 5A-10 of International Patent Application Publication No. WO201/9009710, FIGS. 11E-14B, 17B, and 19A-20D of International Patent Application Publication No. WO2020080938, FIG. 9B of International Patent Application Publication No. WO2020/214027, FIGS. 10-19 of International Patent Application Publication No. WO2023/113598, or several figures in International Patent Application Publication No. WO2023/287289 and International Patent Application Publication No. WO2025026670, each of which is incorporated in its entirety herein by this reference.

In some embodiments, the inner member 110, the intermediate member 120, and/or the outer member 130 may include one or more tendons configured to directly engage a portion of the same elongate member and/or a radially adjacent elongate member to lock the tube assembly in a particular geometry and/or bend angle. Additional details of locking tendons are described herein.

The inner member 110, intermediate member 120, and/or outer member 130 may be formed from any suitable rigid material such as stainless steel, cobalt-chromium, shape memory alloy such as Nitinol®, plastic, polymer, composites and/or other materials. Additionally or alternatively, the elongate member(s) (e.g., inner member 110, intermediate member 120, and/or outer member 130) can be made by a 3D printing process or other known material deposition processes.

During manufacturing, an individual tube may be cut (e.g., laser cut) according to a predetermined pattern. In some embodiments, various features of the elongate members (e.g., inner member 110, intermediate member 120, and/or outer member 130) of the tube assembly 100 (e.g., cut patterns at the flexible regions, tendons, sliders, slots, spacers, etc.) may be formed through removal of material from the wall of each respective tubes forming the members of the tube assembly 100. For example, starting from a cylindrical tube with desired inner and outer diameters (and desired wall thickness), various features of an elongate member (e.g., inner member 110, intermediate member 120, outer member 130) may be formed by removing parts of the wall of the cylindrical tube, such as by laser cutting or water cutting. However, in some embodiments the elongate members may be formed through injection molding, plating techniques, 3D printing or other material deposition process, photochemical etching, deep pressing, conventional chipping techniques such as drilling or milling, and/or any suitable technique. In some embodiments, removal of material may be performed through laser cutting, which enables accurate and clean removal of material under reasonable economic conditions. Moreover, forming each elongate member (e.g., inner member, intermediate member, outer member) of the articulable tube assembly from a respective tube (e.g., metal tube, such as stainless steel) may enable each separate elongate member to maintain a relatively stable tubular form (e.g., flexible, but manipulable similar to a solid tube).

In some embodiments, the predetermined laser-cut pattern may include one or more fracture elements that comprise small segments of uncut material extending across a slit and connecting adjacent portions of the tube. FIG. 6, for example, shows a portion of a tube with fracture elements 148 attaching first sidewall portions 150 with second sidewall portions 152. These fracture elements are designed to bridge the slits between adjacent features to hold them together while the tube remains in a relatively straight configuration, for example while being inserted into another tube or tube assembly or while receiving insertion of another tube or tube assembly. The fracture elements are further configured to break when the tube is subject to a threshold amount of deformation, such as the tube being bent in one or more locations along its longitudinal axis. Breaking of the fracture elements allows relative movement between the features previously connected by the fracture elements. Thus, the fracture elements are configured to remain intact—and thus the features held in a certain arrangement—while the various tubes are inserted in or over one another to form the tube assembly. Having the features in known positions during the assembly process can be helpful for aligning and connecting various features located in different tubes. Once the tubes are fully assembled into a tube assembly and one or more welds are applied to connect various portions of the tubes, the tube assembly may be flexed or otherwise deformed to break the fracture elements and allow relative movement between the features.

The above-mentioned processes may be convenient ways to form each of the inner member 110, the intermediate member 120, and/or the outer member 130 in one overall process, without requiring additional steps for connecting different features of each elongate member. This simplified manufacturing process is advantageously contrasted from multiple steps that are required in manufacturing conventional steerable instruments such as with conventional steering cables, as steering cables must be connected in some way at end regions of a steerable catheter.

The inner and/or outer diameters of the members 110, 120, 130 may be selected such that at any given location along the assembled tube assembly 100, the outer diameter of the inner member 110 is slightly less than the inner diameter of the intermediate member 120, the outer diameter of the intermediate member 120 is slightly less than the inner diameter of the outer member 130, in such a way that a sliding movement of the adjacent members with respect to each other is possible. The dimensioning should be such that a sliding fit is provided between adjacent elongate members. A clearance between adjacent elongate members may generally be in the order of 0.02 to 0.1 mm, but may depend on the specific application and material used. The clearance may be smaller than a wall thickness of the tendons 126 to prevent an overlapping configuration thereof. Restricting the clearance to about 30% to 40% of the wall thickness of the tendons 126 may, for example, be generally sufficient.

Specific dimensions and features of the members 110, 120, and 130 (and/or other elongate members) may vary depending on the application in which the tube assembly 100 may be used. For example, the tube assembly may have a longer flexible portion which may help facilitate the use of the tube assembly in areas of a human body that are navigable in tortuous spaces (e.g., colon, esophagus, airways, curved blood vessels, etc.).

### II. Selected Examples of Helical Structures

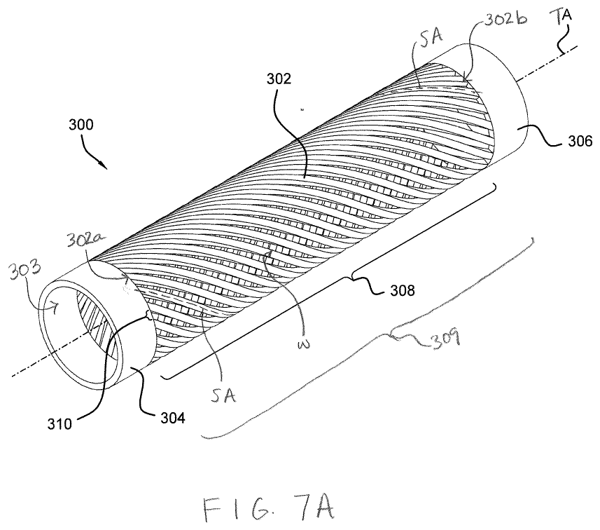

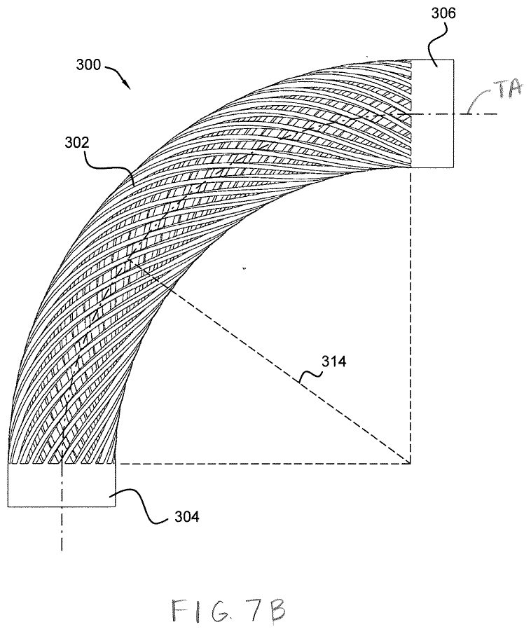

FIG. 7A is a perspective view of a helical structure 300 in a straight configuration and FIG. 7B is a side view of the helical structure 300 in a curved configuration. As shown, the helical structure 300 comprises a tubular sidewall defining a lumen 303 and having a central longitudinal axis TA. The sidewall can be formed of a plurality of helically arranged strands 302 (or “strands 302”) disposed between a first rigid band 304 and a second rigid band 306. As discussed in greater detail below, the helical structure 300 is flexible along the longitudinal axis TA and configured to bend omnidirectionally while maintaining a substantially constant diameter and providing substantially uniform force distribution under both axial compression and tension.

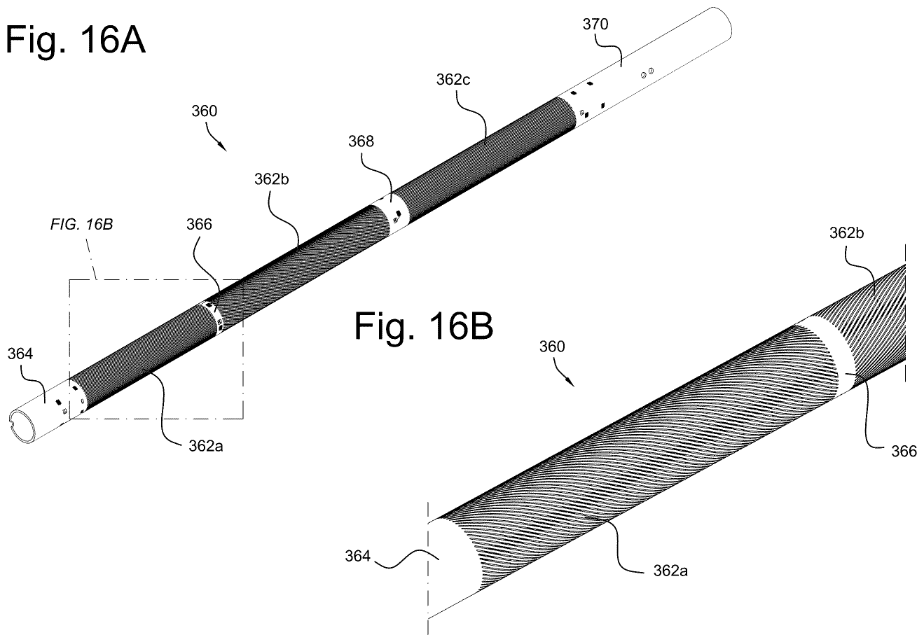

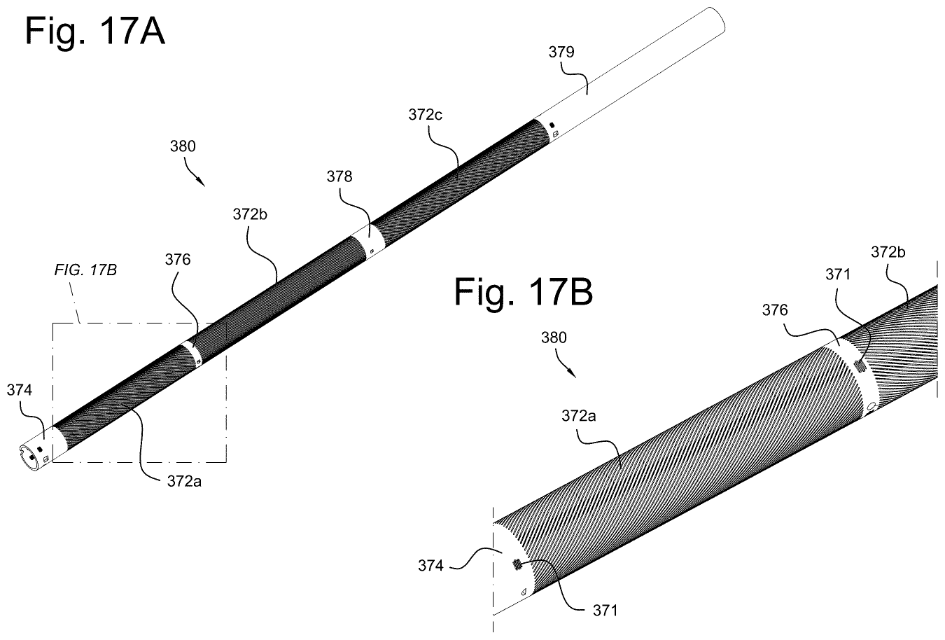

In some embodiments, the helical structure 300 may comprise a shorter tube with only a helical cut pattern (as shown in FIGS. 7A and 7B) and configured for incorporation as one of the tubular layers of a tube assembly 100 of the present technology. In such embodiments, the helical structure 300 can have a length shorter than (e.g., >50% shorter than) one, some, or all of the other elongate tubular members of the tube assembly 100. For example, the helical structure 300 may be incorporated only at or near the distal portion 100b and terminate proximally along the distal portion 100b or a distal region of the intermediate portion 100c. In other embodiments, the helical structure 300 may be a portion of an elongate tubular member (such as any of the elongate tubular members disclosed herein) of the tube assembly 100. In such cases, the first band 304 is integral with or otherwise coupled to (e.g. formed of the same tubular sidewall as) another portion of the elongate tubular member and/or the second band 306 is integral with or otherwise coupled to (e.g. formed of the same tubular sidewall as) another portion of the elongate tubular member. In several of such embodiments, the elongate tubular member may comprise a plurality of helical structures 300 along its length (for example as shown in FIGS. 16A-16B and 17A-17B), separated by rigid bands. Alternatively, the elongate tubular member may include only a single helical structure 300. In such embodiments, the elongate tubular member may not include other cuts or cut patterns (e.g., the remainder of the tube comprises a substantially continuous tubular sidewall) or may include one or more other cuts or cut patterns along its length for other purposes, as detailed herein. In any case, and as described in greater detail below, it may be especially advantageous to align the helical structure 300 with a flexible region of another elongate member of a tube assembly of the present technology (steerable or not), including those flexible regions coinciding with and forming one or more of the steerable regions 102. Such alignment of the helical structure 300 with one or more flexible regions provides structural reinforcement and load-bearing capacity—including resistance to axial and torsional loads imposed by steering tendons and end effectors—while preserving the omnidirectional flexibility and small bend radius capability of the steerable region 102.

Each of the helical strands 302 can wrap helically around the longitudinal axis TA and are separated from one another by helical slots 310. The helical strands 302 may be formed, for example, by removing material from a tube where the remaining material comprises the helical strands 302 and first and second bands 304, 306, and the areas of material removal coincide with the slots 310. As such, each of the helical strands 302 has a first end 302a integral with the first band 304 and a second end 302b integral with the second band 306. The helical structure 300 can comprise any number of helical strands 302, such as two or more, three or more, four or more, five or more, six or more, seven or more, eight or more, nine or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, etc. The number of strands 302 may depend on a desired flexibility. Some, none, or all of the helical strands 302 may be arranged equidistantly.

Each of the strands 302 has a thickness measured in the radial direction. In some embodiments, one, some, or all of the strands 302 have a thickness that is the same as that of the sidewall of the tube from which it is cut. The strands 302 can have the same or different thicknesses. A strand thickness can be in a range of about 0.03 mm to about 2.0 mm, about 0.03 mm to about 1.0 mm, about 0.05 mm to about 0.5 mm, or about 0.08 mm to about 0.4 mm.

Each of the strands 302 has a width w measured perpendicular to the respective strand's longitudinal axis SA. The strands 302 can have the same or different widths. One, some, or all of the strands 302 can have a uniform width along their respective lengths, or may have a width that varies along the strand longitudinal axis SA and/or helical structure longitudinal axis TA. A strand width may depend on the diameter of the tube and the desired bend radius (denoted by 314 in FIG. 7B). For example, a strand width that is too large can be too stiff to bend over a small radius (e.g., it deforms plastically and will fatigue). In some embodiments, the strand width may be in a range of 0.005 mm to about 2 mm, and/or about 4-5% of the diameter of the tube. For example, in some embodiments the strands 302 can have a width of e.g., 0.1 mm for a tube having an outer diameter of 2.1 mm. Other examples are possible. A width of the slots 310 between adjacent strands 302 may be in a range of about 0.01 mm to about 3 mm. Moreover, the helical structure 300 can have an outer diameter in a range of about 1 mm to about 20 mm.

In some embodiments, the helical structure 300 may include one or more spacers (as discussed above with reference to FIG. 5) disposed in one, some, or all of the slots 310 between adjacent strands 302 to reduce the risk of the strands 302 buckling along their longitudinal axes SA (labeled in FIG. 7A). The helical structure 300 can include a spacer in one, some, or all of the slots 310. The spacer can comprise a single strip of material or multiple, spaced apart pieces of material, in each case cut from the same tubular sidewall as the helical strands 302. The spacer(s) can extend from (e.g., is integral with) one of the two adjacent helical strands it is spacing apart such that the helical strand 302 from which it extends has a thickness t greater than along a portion of the helical strand 302 not including a spacer. The spacers can be included in every slot, every other slot, every third slot, a random distribution of slots, or other arrangements.

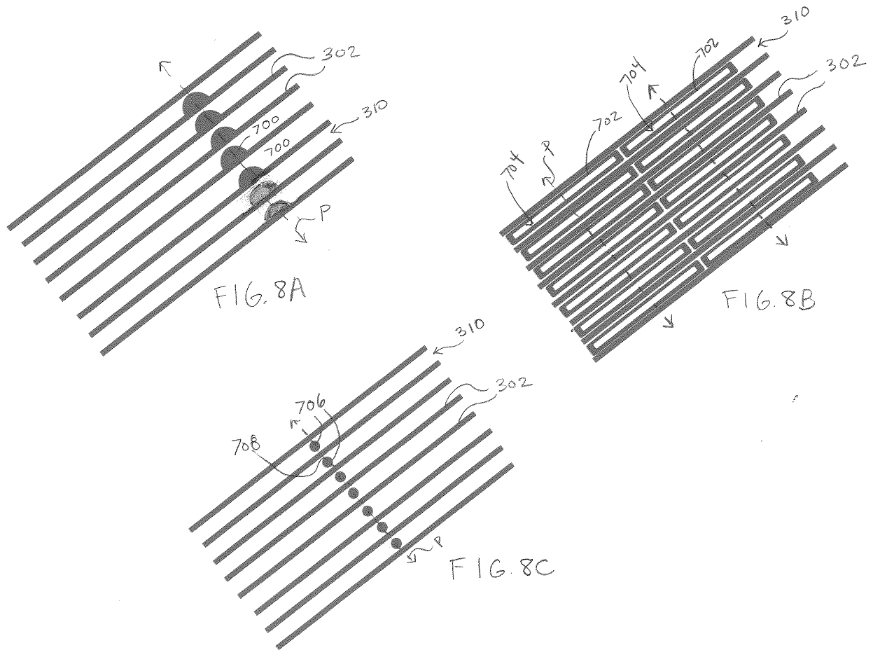

FIGS. 8A-8C show example spacer configurations in accordance with several embodiments of the present technology. In each of FIG. 8A-8C, only a portion of a subset of strands 302 are shown for ease of illustration. As shown in FIG. 8A, in some embodiments the helical structure 300 can include one or more spacers 700 disposed in one, some, or all of the slots 310. The spacer(s) 700 can comprise a protrusion on a respective strand 302 that has a curved surface facing the adjacent strand 302 and separated from the adjacent strand 302 by a gap, at least when the helical structure 300 is in a straight configuration. In some embodiments the spacers 700 can have a semi-circular shape, or any other shape. The spacers 700 may be disposed along their respective strands 302 along a helical path P wrapping in a circumferential direction opposite that of the strands 302. Other arrangements are possible. For instance, in some examples the spacers 700 are not aligned and/or can have other spatial distributions. Additionally or alternatively, the spacers 700 can have the same shape or different shapes.

As shown in FIG. 8B, in some embodiments the helical structure 300 can include one or more rectangular spacers 702 disposed in one, some, or all of the slots 310. Unlike the examples of spacer 146 and 700, the spacers 702 are not attached to and/or do not extend from a strand 302. Instead, the spacers 702 are free-floating within the slots 310. During manufacturing, the spacers 702 may initially be attached to one or both adjacent strands 302 via one or more fracture elements (as described above with reference to FIGS. 7A and 7B), which are subsequently broken to free the spacer 702. In some cases the spacers 702 do not remain free-floating at the end of manufacturing, however, as a polymer is reflowed over the helical structure 300 and extends into the slots 310. In some implementations, for example as shown in FIG. 8B, the spacer 702 can include one or more openings 704. The openings 704 can be especially beneficial when a polymer or other reflowable material is reflowed over the helical structure 300 as they allow the polymer to flow between the gaps while maintaining the desired strand spacing. As a result, the spacer configuration of FIG. 8B can have a greater bending and torsional stiffness than that of FIG. 8A. It will be appreciated that the spacer 702 can have shapes other than that of a rectangle (e.g., square, ovular, circular, triangular, etc.), so long as the shape is large enough to enclose an opening formed by a tube-cutting device. In some embodiments, some or all of the spacers 702 are positioned along their respective strands 302 such that together they form a helical path P wrapping in a circumferential direction opposite that of the strands 302. Other arrangements are possible. For instance, in some examples the spacers 702 are not aligned and/or can have other spatial distributions. Additionally or alternatively, the spacers 702 can have the same shape or different shapes.

As shown in FIG. 8C, in some embodiments the helical structure 300 can include one or more circular spacers 706 disposed in one, some, or all of the slots 310. In some embodiments, the spacers 706 can be free-floating within the slots 310. During manufacturing, the spacers 706 may initially be attached to one or both adjacent strands 302 via one or more fracture elements 708 (as described above with reference to FIG. 6), which are subsequently broken to free the spacer 706. In some cases the spacers 706 do not remain free-floating at the end of manufacturing, however, as a polymer is reflowed over the helical structure 300. It will be appreciated that the spacer 706 can have shapes other than that of a circle, such as rectangular, square, ovular, triangular, etc. In some embodiments, some or all of the spacers 706 are positioned along their respective strands 302 such that together they form a helical path P wrapping in a circumferential direction opposite that of the strands 302. Other arrangements are possible. For instance, in some examples the spacers 706 are not aligned and/or can have other spatial distributions. Additionally or alternatively, the spacers 706 can have the same shape or different shapes.

Referring again to FIGS. 7A and 7B, an axial length of the portion of the helical structure 300 along which the helical strands 302 extend 360 degrees or an integer multiple thereof (e.g., 1 time or 360 degrees, 2 times or 720 degrees, 3 times or 1080 degrees, etc.) is referred to herein as a “helical length 308.” As such, for a helical length 308, the first ends 302a of each of the strands 302 at the first band 304 are disposed at substantially the same circumferential position as the second ends 302b at the second band 306. As used herein, the term “substantially” is to be understood as meaning “exactly” within manufacturing tolerances, e.g., within plus or minus 5%. In the embodiment shown in FIGS. 7A and 7B, the strands 302 wrap 360 degrees along the helical length 308, though as noted above other integer values of 360 degrees are possible. In some embodiments, the helical length 308 may be in a range of about 5 mm to about 100 mm. Other lengths are possible.

An axial distance between the first and second bands 304, 306 is referred to herein as a “band length 309.” In some embodiments, for example as shown in FIGS. 7A and 7B, the helical length 308 and the band length 309 are substantially the same. A configuration in which the helical length 308 and the band length 309 are the same (regardless of how many full rotations the strands 302 make) minimizes and/or reduces path length differences between the strands 302 when the helical structure 300 is bent along the longitudinal axis TA. Likewise, the helical structure 300 maintains substantially the same band length 309 whether bent or straight. As a result, the bent strands 302 remain located at substantially the same radial distance of the central longitudinal axis TA along their lengths, similar to the unbent configuration of the helical structure 300 shown in FIG. 7A. This provides a substantially uniform outer and inner diameter even when the helical structure 300 is bent, even when the helical structure 300 has a small bend radius. Without the radial deviations caused by path length differences, the strands 302 do not impede structures in radially adjacent tubes (e.g., arranged inside or outside the helical structure 300). Moreover, because each of the strands 302 of the helical structure 300 have portions on both the outer and inner radii of curvature, the strands 302 undergo equal stretching and compression which prevents internal stress and material strain from path length differences. These advantages are especially beneficial when the helical structure 300 is coaxial with and axially aligned with a steerable region(s) 102, which experience high axial and torsional loads during use while needing to retain the ability to bend into a certain bend radius. The helical structures 300 of the present technology fortify the steerable region 102 and provide the additional axial and torsional stiffness while maintaining a desired level of flexibility at the steerable region 102.

The slot width, strand width w, strand thickness t, and number of strands 302 can affect the flexibility and stiffness of the helical structure 300. The slope of the strands 302 can be selected such that the helical strands 302 cover 360 degrees, or an integer multiple thereof, around the tube at exactly the band length 309. Because changing the slope of the strands 302 impacts the helical structure's resistance to bending (e.g., the greater the slope, the greater the resistance to bending), the strand width w can be adjusted to counteract the increased resistance when adjusting the slope.

As discussed in greater detail below, in some implementations a helical structure can be used to provide additional axial stiffness for a flexible region of a radially adjacent tubular layer. In such cases, however, it may be beneficial to allow the strands 302 to extend more than 360 degrees (or an integer multiple thereof) between the first and second bands 304, 306. FIG. 9A, for example, is a side view of another helical structure 800 in accordance with several embodiments of the present technology, shown with a flexible structure 850 of a radially adjacent tubular layer (shown separated from the helical structure 800 for ease of explanation). The flexible structure 850 includes a flexible region 858 bound axially between first and second rigid bands 854, 856. Here, the strands 302 of the helical structure 800 extend axially beyond the helical length 308 by an extra length 312 such that the band length 309 is slightly greater than the helical length 308. This further extension of the strands 302 may additionally or alternatively be applied at the other axial end of the strands 302 (nearer the first band 304). Such a configuration can have several benefits when the helical structure is coaxial with another tubular layer and axially aligned with a flexible region of that layer. For example, the extra length 312 can provide a bending stiffness transition at the ends of the strands 302. While the helical structures herein can generally have a uniform bending stiffness along their lengths, in some cases the helical structures can have a higher stiffness where the strands 302 meet the bands 304, 306. These higher-stiffness transition regions can affect the bending performance of an overlapping flexible region of an adjacent tube. As such, it may be beneficial to include the extra length 312 as a buffer zone at one or both ends of the helical structure such that the flexible region only overlaps with the helical length 308, and the rigid band(s) 856 of the flexible structure 850 prevent the extra length 312 from bending, thereby making the helical length 308 the only functional length of the strands (and thus preserving path length compensation).

A helical structure incorporating the extra length 312 at one or both ends can also be beneficial when the entire helical structure is configured to move axially relative to a radially adjacent tube containing an overlapping flexible region, as depicted by FIG. 9B. For example, in some cases the helical structure 800 may be a portion of a longer tube that is used as a push/pull member to actuate an end-effector coupled to the distal end of the tube. In such cases, the push/pull helical structure tube can be disposed through the lumen of another elongate tube having a flexible region 858 that is generally axially aligned with the helical structure 800. The flexible region 858 can have a length substantially equal to that of the helical length 308 of the helical structure 800. Before the helical structure 800 moves, the rigid band 856 of the flexible structure 850 is aligned with and preventing the extra length 312 of the helical structure 800 from bending (as shown in FIG. 9A). When the helical structure 800 moves axially relative to the flexible structure 850, as shown in FIG. 9B, some 330 of the extra length 312 is no longer be aligned with the second band 856 and thus is allowed to bend. However, the axial shift also results in a portion 332 of the strands 302 at the opposite end now being aligned with the first rigid band 854, thereby effectively limiting the bendable portion of the helical structure to the helical length 308.

In some applications, an axial load may be applied to the helical structure 300 (or helical structure 800) that causes the diameter of the helical structure 300 to increase (e.g., the helical structure 300 is under axial compression) or decrease (e.g., when the helical structure 300 is under axial tension). For example, the axial load may be applied by a steering tendon 126 (to articulate a steerable region 102), an end effector coupled to the distal end of the elongate member and/or tube assembly, or other axial load. The effects on the diameter of the helical structure can be especially pronounced at or near a mid-portion of the helical structure 300. In such cases, one or more elongate tubular members may be positioned on one or both radial sides (e.g., inside and/or outside) of the helical structure 300 to constrain the helical structure 300 under axial forces and thus enable the helical structure 300 to withstand such loading.

Additionally or alternatively, in some applications the helical structure (e.g., 300 or 800) may be manipulated such that it generates torque, which can lead to unwanted rotational forces. For example, in use the helical structure 300 may be rotated (whether purposely in response to user manipulation or passively in response to a local environment) about its longitudinal axis TA which causes the strands 302 to wind or unwind and thereby occupy more or less radial space. To prevent or mitigate such torque reaction forces, a second helical structure can be coaxially positioned inside or outside the helical structure 300. FIG. 10, for example, shows a helical assembly 900 comprising the helical structure 300 arranged coaxially within a second helical structure 321. Helical structure 321 can be generally similar to helical structure 300, except as noted herein. The helical structure 321 can be a standalone tube or a portion of an elongate tubular member, as described above with reference to helical structure 300. Helical structure 321 has a first rigid band 324 at one longitudinal end and a second rigid band 327 at an opposite longitudinal end, with helical strands 322 extending therebetween along a helical length 328. The strands 322 can wrap around the longitudinal axis TA 360 degrees, or an integer multiple thereof, between the first and second bands 324, 327. The strands 322 may be separated by helical slots 330 as detailed above, and can have the number of strands, strand width, strand thickness, slot width, and optional spacer variations described above.

Helical structure 300 and helical structure 321 may be axially aligned such that their respective helical lengths 308, 328 axially coincide, their respective first bands 304, 324 axially coincide, and their respective second bands 306, 327 axially coincide. First band 304 can be fixed to first band 324 at one or more locations, and second band 306 can be fixed to second band 327 at one or more locations. As such, the helical structures 300, 321 are configured to translate and rotate with one another. The first bands 304, 324 can be fixed to one another and the second bands 306, 327 can be fixed to one another via any suitable mechanical connection, such as welding (e.g., laser-welding), a pen-hole connection, a bayonet connection, gluing, or others.

The helical assembly 900 is flexible and can be bent in a curve, similar to helical structure 300 shown in FIG. 7B. Provided the helical length 328 is the same as helical length 308, the strands 302, 322 will not undergo path length differences caused by the bending and will remain located at substantially the same radial distance of central longitudinal axis A as in the unbent configuration of FIG. 10.

Helical structure 321 differs from helical structure 300 in that strands 322 extend in an opposite helical direction as strands 302. For example, the strands 302 of helical structure 300 can extend in a clockwise direction while the strands 322 of helical structure 321 can extend in a counterclockwise direction, or vice versa. Because helically arranged strands 302 and 322 are wound in opposite helical directions they provide the helical assembly 900 with improved torsional and axial stiffness. The two-layer configuration neutralizes and/or reduces torque reaction forces generated by the individual helical structures 300, 321, thus ensuring controlled and stable bending without twisting. Such features may be especially advantageous when applied to a flexible and/or steerable region of the tube assemblies of the present technology.

In some implementations, the helical assembly 900 includes the helical structure 800 of FIG. 9A arranged coaxially with helical structure 321 (inside or outside). In such embodiments, the helical structures 800, 321 may be positioned such that the helical lengths 308, 328 are axially aligned. If such an embodiment is bent into a curve (like the one shown in FIG. 7B), the second band 327 prevents the portions of the strands 302 inside the extra length 312 from bending. As a result, these portions of the strands 302 will not suffer from path length changes in the curved configuration and will not undergo radial forces.

### III. Example Flexible Structures

Early implementations of flexible sections in articulating instruments commonly relied on creating circumferential or axial slots in the wall of metal tubes, for example as shown in FIG. 11A. These slots x1 locally reduce the bending stiffness of the tube, enabling flexure. Formation of the slots x1 in the tubular sidewall creates bridges x2 extending between axially adjacent bands x3. When the tube is bent, the individual bridges x2 function as elastic beams that bend and flex to enable articulation. This behavior, however, can be undesirable as the bridges x2 absorb the vast majority of bending stresses, thereby concentrating stress on a limited amount of material and increasing susceptibility to fatigue failure.

Another example conventional flexible structure is depicted in FIG. 11B, shown in a straight configuration. Such a structure, referred to herein as an “H-hinge,” includes paired bridges y1 cut within a single plane (spaced 180 degrees from one another), followed by a subsequent pair of bridges y2 cut in a plane rotated by a defined angle (typically 90 degrees) from the axially adjacent bridge pair y1. This repeated arrangement of four bridges y1, y2 enables bidirectional bending in two orthogonal planes. Struts y3 extend circumferentially between bridges of axially adjacent bridge pairs such that the struts y3 are substantially parallel to one another and perpendicular to a longitudinal dimension of the tube. Unlike the flexible structure shown in FIG. 11A, bending of the tube is enabled by bending of the struts y3 rather than the bridges. Such a configuration can be undesirable in certain applications, as it provides limited axial stiffness and can require relatively longer lengths of tube to form a given bend angle, which is especially problematic when sharp turns are required.

The flexible tube architectures of the present technology provide several advantages over the aforementioned conventional designs. As discussed in greater detail below, the flexible structures of the present technology enable elastic deformation in two orthogonal bending planes while providing improved axial stiffness, a more uniform stress distribution along the struts (and thus improved fatigue life), and a smaller overall bend radius for a given length of tube (i.e., less tube length is required to make the same bend radius).

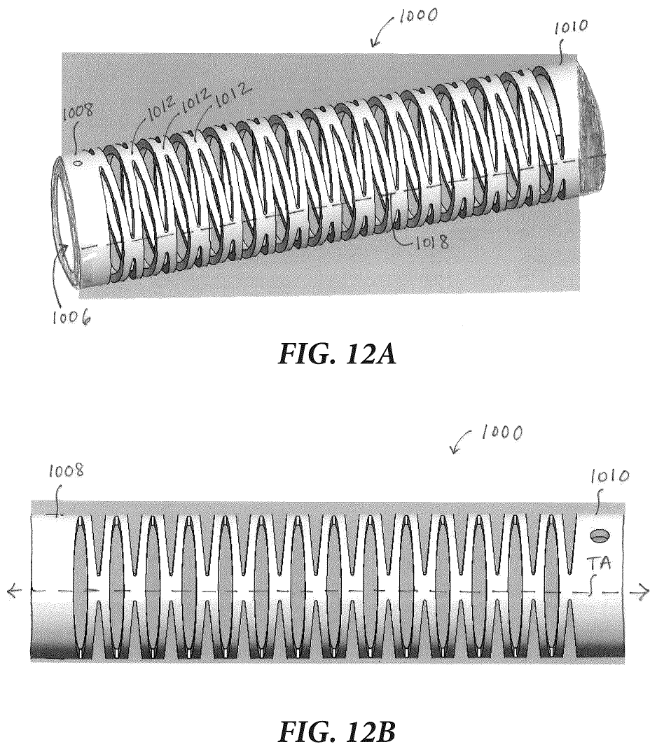

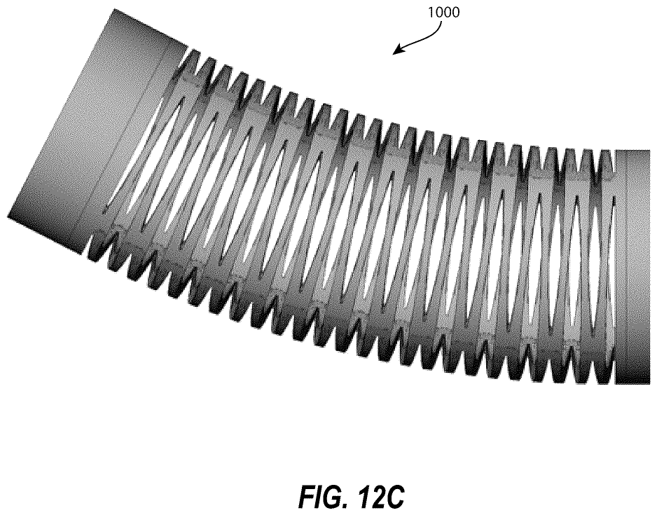

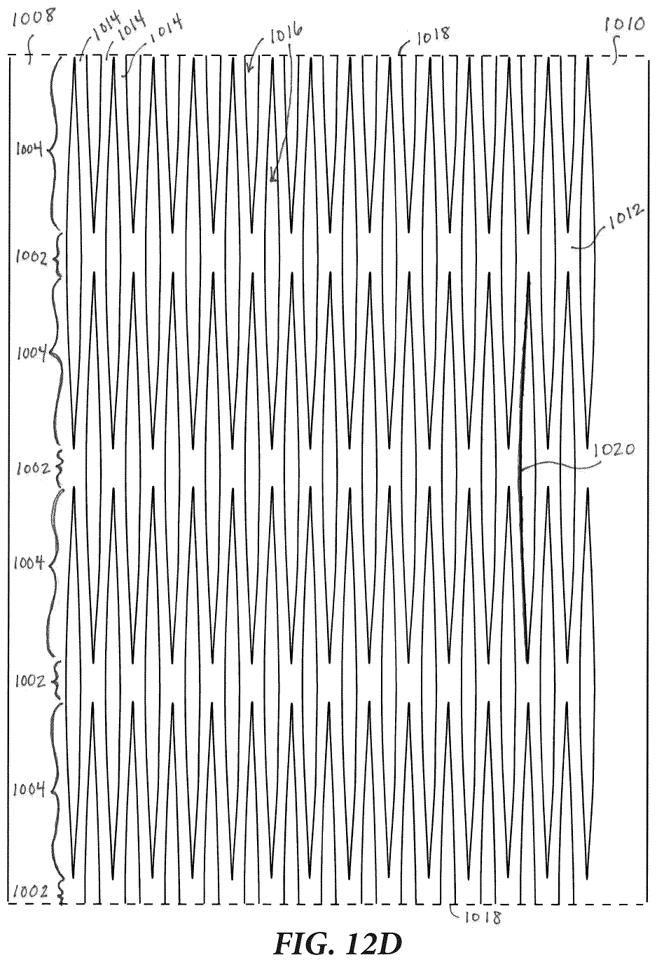

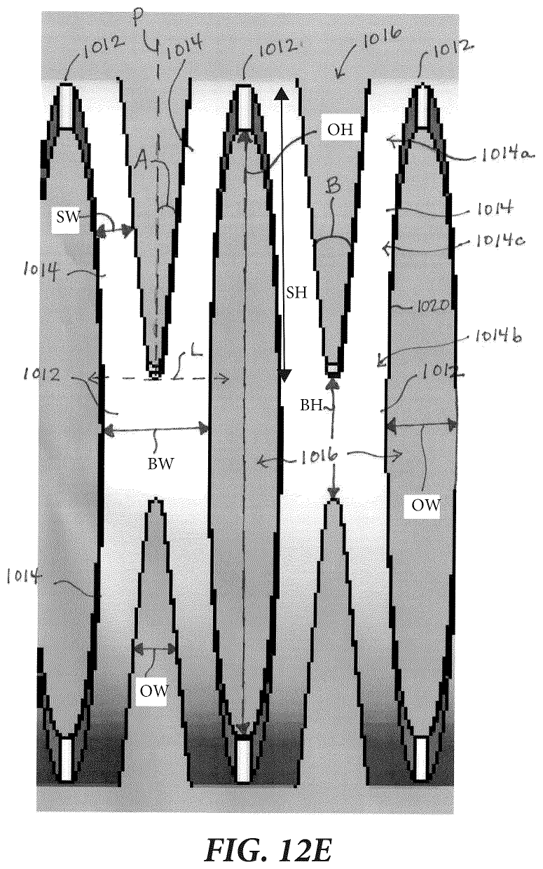

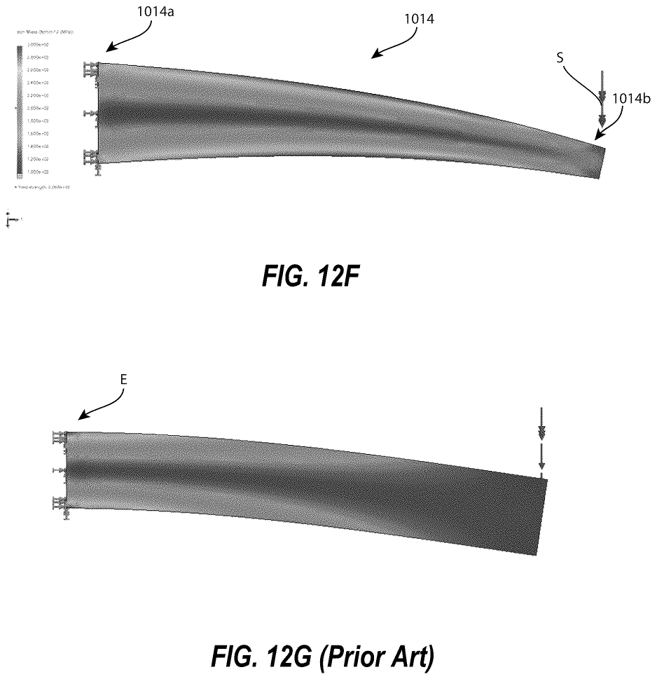

FIGS. 12A and 12B are perspective and side views, respectively, of a flexible structure 1000 configured for use with the tube assemblies of the present technology, shown in a straight configuration. FIG. 12C is a side view of the flexible structure 1000 in a curved configuration. FIG. 12D is a plan view of the flexible structure 1000 if it were cut along line 1018 (see FIG. 12A) and laid flat, and FIG. 12E is an enlarged view of a portion of the flexible structure 1000. With reference to FIGS. 12A-12E together, the flexible structure 1000 comprises a tubular sidewall defining a lumen 1006 and having a central longitudinal axis TA (labelled in FIG. 12B). The sidewall can be formed of a plurality of bridge regions 1002 and a plurality of strut regions 1004 alternating with the plurality of bridge regions 1002 along a circumference of the tube such that the strut regions 1004 extend circumferentially between and connect adjacent bridge regions 1002. Each of the bridge and strut regions 1002, 1004 extend axially between a first rigid band 1008 and a second rigid band 1010. The cut pattern of the flexible structure 1000 can be incorporated along any portion (distal portion, intermediate portion, proximal portion) of the elongate tubular members and/or steerable assemblies disclosed herein. In some cases, it can be especially beneficial to incorporate the flexible structure 1000 along one, some, or all flexible regions that coincide with the steerable region of the tube assembly, and/or any passively flexible portion of the tube assembly. The unique geometries of the bridge and strut regions 1002, 1004 enable omnidirectional bending of the flexible structure 1000, achieving a smaller minimum bend radius over a shorter tube length than conventional designs (including H-hinges) while providing torsional stiffness to radially adjacent tubular structures.

Each of the plurality of bridge regions 1002 comprise a plurality of bridges 1012 that are spaced apart along the longitudinal axis TA by circumferentially extending openings 1016 in the sidewall. In some implementations, the flexible structure 1000 includes pairs of bridges 1012 separated by openings 1016, where each pair of bridges and corresponding openings 1016 lie in a plane orthogonal to the longitudinal axis TA. In such cases, the bridges 1012 may be located at circumferentially opposite locations (e.g., 180 degrees apart) along the flexible structure 1000. Axially adjacent pairs of bridges 1012 and openings 1016 can be rotated 90 degrees from one another, thereby enabling articulation of the flexible structure in multiple planes. It will be appreciated that the bridges 1012 of a given pair of bridges 1012 can have circumferential spacings other than 180 degrees, and axially adjacent pairs of bridges 1012 can be shifted more or less than 90 degrees.

As best shown in FIG. 12E, each of the bridges 1012 has a bridge width BW (e.g., measured along a longitudinal dimension) and a bridge height BH (e.g., measured along a circumferential direction). The bridge heights and widths BH, BW can be selected based on a desired flexibility and/or bend radius of the flexible structure 1000. One, some, or all of the bridges 1012 can have the same or different heights BH, and one, some, or all of the bridges 1012 can have the same or different widths BW. Likewise, each of the openings 1016 has an opening height OH and opening width OW. One, some, or all of the openings 1016 can have the same or different heights OH, and one, some, or all of the openings 1016 can have the same or different widths OW.

One, some, or all of the bridges 1012 along the flexible structure 1000 can be fixed to a radially adjacent (e.g., underlying and/or overlying) tubular layer. For example, one, some, or all of the bridges 1012 may be welded to an axially aligned portion of a radially adjacent elongate tubular member. In these and other embodiments, one, some, or all of the bridges 1012 can be coupled to a radially adjacent tubular layer via other means. For example, in some embodiments, a portion of one, some, or all of the bridges 1012 can extend radially upwardly or downwardly to engage an opening and/or slot in a radially adjacent tubular layer. In some implementations, a portion of one, some, or all of the bridges 1012 can have an opening configured to receive a radially extending portion of a radially adjacent tube. In the latter two implementations, the radial protrusion and the opening can serve as a fixation point for the adjacent tubular layers, or the radial protrusion can be configured to slide within the opening and/or slot.

Each of the plurality of strut regions 1004 comprise a plurality of struts 1014 that extend between circumferentially adjacent bridge regions 1002. Axially adjacent struts 1014 within a respective strut region 1004 may extend from a shared bridge 1012 in the same circumferential direction but in different or opposite axial directions. Said another way, within one, some, or all of the strut regions 1004, at least some of the struts 1014 extend at an angle relative to an axially adjacent strut 1014 such that axially consecutive struts 1014 define a zig-zag configuration. With reference to FIG. 12E, at least when the flexible structure or tube 1000 is in a straight configuration, one, some, or all of the struts 1014 can extend at a non-perpendicular angle A relative to a plane P bisecting the bridge 1012 from which the respective strut 1014 extends, where the plane P is orthogonal to the longitudinal axis TA of the flexible structure 1000. Said yet another way, one, some, or all of the struts 1014 can be angled relative to a length dimension L of the flexible structure 1000. In any case, the angle A may be selected based on a desired axial stiffness, maximum achievable bending angle, and torsional stiffness of the flexible structure 1000. In some cases, the angle A is at least 5 degrees, at least 10 degrees, at least 15 degrees, or at least 20 degrees. The angulation of the struts 1014 enable each strut to function as an elastic beam, contributing simultaneously to bending in a plane parallel to the longitudinal axis TA (FIG. 12B) of the structure 1000, and a plane perpendicular to the longitudinal axis TA.

For a given bend angle and opening width OW, the angled struts 1014 allow for a smaller bridge width BW (as compared to an H-hinge) such that the struts 1014 can be placed closer together along the length of the flexible structure 1000. As a result, the flexible structure 1000 can achieve a desired bend radius using a shorter amount of tube. For example, it may require 9 mm of length of the flexible structure 1000 to form a 90-degree bend, while it would require at least 10 mm of length of the H-hinge to make the same turn, assuming a same tube material, wall thickness, and outer diameter. In some cases, the flexible structure 1000 can achieve a given bend radius, including small bend radii, with 50-90% of the tube length required for the same bend radius for an H-hinge. As used herein, “small bend radius” can refer to a bend radius of 2 mm or less, 3 mm or less, 4 mm or less, 5 mm or less, 6 mm or less, 7 mm or less, 8 mm or less, 9 mm or less, 10 mm or less, 11 mm or less, 12 mm or less, 13 mm or less, 14 mm or less, or 15 mm or less.

An angle B between axially adjacent struts 1014 within a given strut region 1004 can be varied based on a desired bending stiffness and/or axial stiffness of the flexible structure 1000. For example, increasing the angle B between axially adjacent struts 1014 (while keeping the strut lengths the same) increases the bending stiffness and axial stiffness while decreasing the angle B decreases the bending stiffness and axial stiffness.

Referring still to FIGS. 12A-12E, one, some, or all of the struts 1014 may curve as they extend circumferentially between adjacent bridges 1012 such that the axial sides 1020 of the struts 1014 (one labeled in each of FIGS. 12D and 12E) are concave in either a first axial direction or a second axial direction. Such curvature beneficially distributes bending stresses along the length of the respective struts 1014, with increased flexibility at or near a midpoint of a given strut 1014, thereby reducing peak stress concentrations. FIG. 12F, for example, shows a finite element analysis (FEA) of half of a strut 1014 being deformed under stress S. The end labeled 1014a corresponds to the end at or adjacent a bridge 1012, while the end labeled 1014b corresponds to a mid-point of the strut 1014. As shown, stress S is distributed substantially uniformly along the length of the strut 1014. The other half of the strut 1014 (not shown) experiences substantially the same distribution. This is in contrast to the force distribution of a strut of an H-hinge, which is more concentrated towards the end E of the strut at the bridge (as shown in FIG. 12G) and more prone to fatigue failure.

In some embodiments, one, some, or all of the struts 1014 can have first portions 1014a, 1014b (one strut labeled in FIG. 12E) at or near a corresponding bridge 1012 and second portions 1014c extending between the first portions 1014a, 1014b, generally disposed at or near a middle portion of the strut 1014. In some embodiments, the individual first portions 1014a, 1014b can have widths greater than the width of the second portion 1014c. In some embodiments, the strut widths SW may continuously taper from the ends of the struts (at the bridges) towards the middle of the strut.

In some embodiments, one, some, or all of the bridges 1012 may connect to four struts 1014. In some cases, a first one of the connecting struts 1014 extends away from the respective bridge 1012 in a first circumferential direction and a first axial direction, a second one of the connecting struts 1014 extends away from the respective bridge in a first circumferential direction and a second axial direction opposite the first axial direction, a third one of the connecting struts 1014 extends away from the respective bridge 1012 in a second circumferential direction opposite the first circumferential direction and in the first axial direction, and a fourth one of the connecting struts 1014 extends away from the respective bridge 1012 in the second circumferential direction and the second axial direction.

Referring still to FIG. 12E, each of the struts 1014 can have a strut width SW (e.g., measured perpendicular to the strut axis) and a strut height SH (e.g., measured in a circumferential dimension). The strut width SW and strut height SH can be selected based on a desired flexibility and/or bend radius of the flexible structure 1000. One, some, or all of the struts 1014 can have the same or different strut widths SW. Likewise, one, some, or all of the struts 1014 can have the same or different strut heights SH. The strut width SW can be the same or different across different strut regions 1004 depending on a desired bending and/or stiffness profile. The strut height SH can be the same or different across different strut regions 1004 depending on a desired bending and/or stiffness profile.

The flexible structure 1000 shown in FIGS. 12A-12E includes four bridge regions 1002 and four strut regions 1004. Other configurations are possible. For example, in some implementations the flexible structure 1000 includes at least two bridge regions 1002 and at least two strut regions 1004, at least three bridge regions 1002 and at least three strut regions 1004, at least five bridge regions 1002 and at least five strut regions 1004, at least six bridge regions 1002 and at least six strut regions 1004, etc.

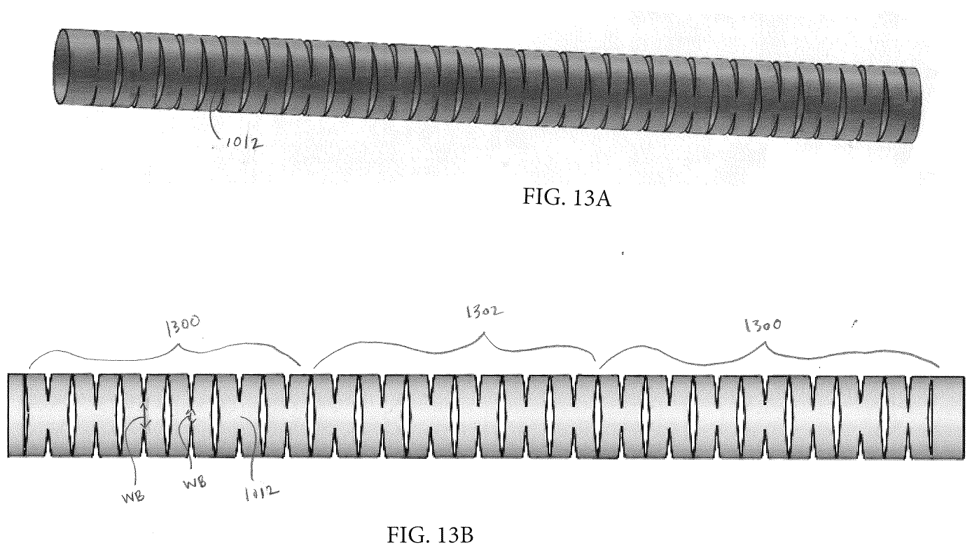

In some cases, for example as shown in FIGS. 13A and 13B, the bridge height BH may alternate for axially consecutive bridges 1012 along all or a portion of a given bridge region 1002. A flexible structure can have end portions 1300 with bridges 1012 having alternating bridge heights BH and an intermediate portion 1302, positioned axially between the end portions 1300, that has a plurality of bridges 1012 with substantially constant bridge heights BH. The bridge height BH can be the same or different across different bridge regions 1002 depending on a desired bending and stiffness profile. The bridge width BW can be the same or different across different bridge regions 1002 depending on a desired bending and stiffness profile.

In some cases the flexible structure 1000 may be coaxially positioned inside or outside any of the helical structures and/or other flexible structures disclosed herein, and others, as detailed below in Section IV. In some embodiments, the flexible structure 1000 may be incorporated within a tube assembly such that the first and second bands 1008, 1010 axially coincides with the bands of one or more helical structures of other tubular layers (e.g., helical structure 300, 321, 800, etc.). In some embodiments, the flexible structure 1000 may be coaxially positioned around or inside only a single helical structure. In some implementations, the flexible structure 350 may be radially positioned between two helical structure (e.g., helical structure 300 and helical structure 321). The wall thickness of the flexible structure 1000 may be more than, less than, or the same as one, some, or all of the tubes of the helical structures.

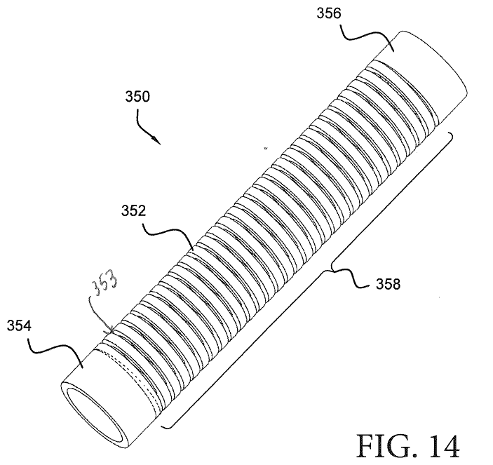

FIG. 14 shows another flexible structure 350 for use the with elongate tube members and/or tube assemblies of the present technology. The flexible structure 350 comprises a first rigid band 344, a second rigid band 346, and a continuous strip 352 extending therebetween. The strip 352 may be formed by providing a single continuous slit 353 in the sidewall of the tube, thereby forming a helical spring. Such a configuration enables omnidirectional bending. The strip 352 can extend axially between the first and second rigid bands 354, 356 along a band length 358.

In some cases, the flexible structure 350 may be coaxially positioned inside or outside any of the helical structures and/or other flexible structures disclosed herein, and others, as detailed below in Section IV. In some embodiments, the flexible structure 350 may be incorporated within a tube assembly such that the helical length 358 axially coincides with the helical length 308 of helical structure 300 and helical length 328 of helical structure 321. In some embodiments, the flexible structure 350 may be coaxially positioned around or inside only a single helical structure. In some implementations, the flexible structure 350 may be radially positioned between two helical structures (e.g., helical structure 300 and helical structure 321). The wall thickness of the flexible structure 350 may be more than, less than, or the same as one, some, or all of the tubes of the helical structures.

### IV. Example Tube Assemblies

While the unique geometry of the flexible structure 1000 provides several advantages over conventional flexible tube designs, the amount of material removed and arrangement of the struts greatly reduces the axial stiffness of the structure 1000. In some cases it may be beneficial to provide the flexible structure 1000 with additional axial stiffness via incorporation of one or more radially adjacent elongate tubular members. FIG. 15, for example, shows a variation of the flexible structure 1000, coaxially positioned with helical structures 300, 321 described above with reference to FIGS. 7A-10. As shown, the rigid bands at either end of the flexible and helical structures can be axially aligned and in some cases fixed to one another (e.g., via welding or other means). As such, the helical structures 300, 321 can provide additional axial stiffness to the flexible structure 1000 and the flexible structure 1000 can provide additional torsional stiffness to the helical structures 300, 321.

While the flexible structure 1000 is shown coaxially surrounding helical structures 300, 321, in other embodiments the helical structures 300, 321 may coaxially surround flexible structure 340. In some examples, the flexible structure 1000 may be positioned radially between the helical structures 300, 321. In any case, one or more other tubular structure may be positioned between the helical structures 300, 321 and the flexible structure 1000. In some embodiments, the flexible structure 1000 may be used with only one of the helical structures, which may be positioned outside or inside the flexible structure 1000, with or without another tubular layer therebetween. A wall thickness of the flexible structure 1000 and a wall thickness of the helical structure(s) can be the same or different. In some embodiments, the helical structures 300, 321 can have individual wall thicknesses that are half that of the flexible structure 1000 such that the wall thicknesses of the helical structures 300, 321 together amounts to the wall thickness of the flexible structure 1000.

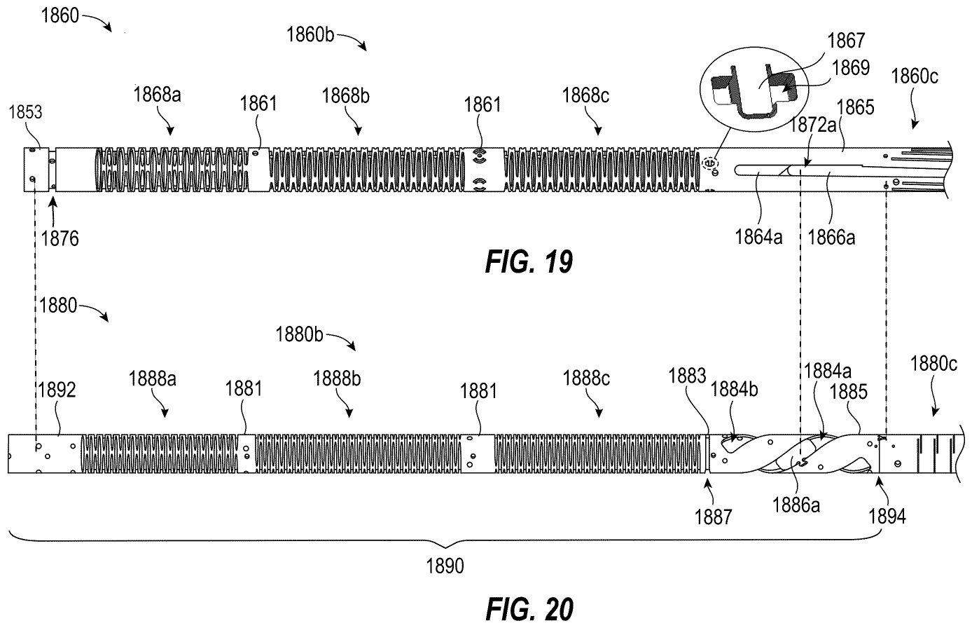

Some tube assemblies of the present technology may include at least two coaxially arranged elongate tubular members, each having at least one helical structure along its length. The elongate members may be arranged such that the helical structures and rigid bands are axially aligned, as previously discussed. Said tube assembly can include one or more other elongate tubular members coaxially disposed outside and/or inside of the assembly. A disassembled example of such a tube assembly is shown in FIGS. 16A-16B and FIGS. 17A-17B. FIGS. 16A-16B show a first elongate member 360 of the tube assembly and FIGS. 17A-17B show a second elongate member 380 of the tube assembly. The first elongate member 360 may be positioned outside of the second elongate member 380 or vice versa. As used herein, “helical tube assembly” refers to the first and second elongate members 360, 380 (or variations thereof) when assembled. The helical tube assembly can further include one or more additional elongate tubular members (such as that shown in FIGS. 19A-19B, or others) disposed inside and/or outside the helical tube assembly.

As shown in FIGS. 16A-16B, the first elongate member 360 can comprise three helical structures (referred to as first helical structure 362a, second helical structure 362b, and third helical structure 362c). More or fewer helical structures are possible (e.g., one helical structure, two helical structures, four helical structures, etc.). In some embodiments, including that shown in FIGS. 16A and 16B, the strands of the first and third helical structures 362a, 362c wind in the same circumferential direction, while the strands of the second helical structure 362b wind in an opposite circumferential direction of the first and third helical structures 362a, 362c. As such, the wind direction of axially adjacent helical structures may alternate. In some embodiments, all the helical structures of a given elongate member can wind in the same direction. One, some, or all of the helical structures 362a, 362b, 362c can have a helical length substantially equal to the band length, as discussed herein with reference to FIGS. 7A and 7B. In some cases, one, some, or all of the helical structures 362a, 362b, 362c can have an extra length such that the helical length is not equal to the band length, as discussed above with reference to FIGS. 9A and 9B.

In any case, the first elongate member 360 can include first, second, third and fourth rigid portions 364, 366, 368, and 370 of the sidewall, with the second and third rigid portions 366, 368 disposed between the first and second helical structures 362a, 362b and the second and third helical structures 362b, 362c, respectively. The first rigid portion 364 is positioned distal of the first helical structure 362a and in some cases may comprise the distal-most portion of the first elongate member 360, as shown. The fourth rigid portion 370 is positioned proximal of the third helical structure 362c and in some cases may comprise the proximal-most portion of the first elongate member 360. In some embodiments, the first elongate member 360 extends beyond the fourth rigid portion 370 and may include one or more flexible regions that coincide with an intermediate portion 100c of a tube assembly in which the first elongate member 360 is incorporated.

As shown in FIGS. 17A and 17B, the second elongate member 380 can comprise three helical structures (referred to as first helical structure 372a, second helical structure 372b, and third helical structure 372c). More or fewer helical structures are possible (e.g., one helical structure, two helical structures, four helical structures, etc.). In some embodiments, including that shown in FIGS. 17A and 17B, the strands of the first and third helical structures 372a, 372c wind in the same circumferential direction, while the strands of the second helical structure 372b wind in an opposite circumferential direction of the first and third helical structures 372a, 372c. As such, the wind direction of axially adjacent helical structures may alternate. In some embodiments, all the helical structures of a given elongate member can wind in the same direction. One, some, or all of the helical structures 372a, 372b, 372c can have a helical length substantially equal to the band length, as discussed herein with reference to FIGS. 7A and 7B. In some cases, one, some, or all of the helical structures 372a, 372b, 372c can have an extra length such that the helical length is not equal to the band length, as discussed above with reference to FIG. 9A.

In any case, the second elongate member 380 can include first, second, third and fourth rigid portions 374, 376, 378, and 379 of the sidewall, with the second and third rigid portions 376, 378 disposed between the first and second helical structures 372a, 372b and the second and third helical structures 372b, 372c, respectively. The first rigid portion 374 is positioned distal of the first helical structure 372a and in some cases may comprise the distal-most portion of the second elongate member 380, as shown. The fourth rigid portion 379 is positioned proximal of the third helical structure 372c and in some cases may comprise the proximal-most portion of the second elongate member 380. In some embodiments, the second elongate member 380 extends beyond the fourth rigid portion 379 and may include one or more flexible regions that coincide with an intermediate portion 100c of a tube assembly in which the second elongate member 380 is incorporated.

In some cases, the first rigid portion 364 of the first elongate member 360 can be longer than the first rigid portion 374 of the second elongate member 380, or they may have the same length. Likewise, the second rigid portions 366, 376 can have the same or different lengths, the third rigid portions 368, 378 can have the same or different lengths, and the fourth rigid portions 370, 379 can have the same or different lengths. Moreover, the first helical structures 362a, 372a can have the same or different lengths, the second helical structures 362b, 372b can have the same or different lengths, and the third helical structures 362c, 372c can have the same or different lengths.

When the first and second elongate members 360, 380 are assembled such that they are coaxially positioned, the first rigid portions 364, 374 can be axially aligned, the first helical structures 362a, 372a can be axially aligned, the second rigid portions 366, 376 can be axially aligned, the second helical structures 362b, 372b can be axially aligned, the third rigid portions 368, 378 can be axially aligned, the third helical structures 362c, 372c can be axially aligned, and the fourth rigid portions 370, 379 can be axially aligned. The first rigid portions 364, 374 may be fixed to one another (e.g., via one or more laser welds), the second rigid portions 366, 376 may be fixed to one another (e.g., via one or more laser welds), the third rigid portions 368, 378 may be fixed to one another (e.g., via one or more laser welds), and the fourth rigid portions 370, 379 may be fixed to one another (e.g., via one or more laser welds).

The first and second elongate members 360, 380 can be arranged such that radially adjacent helical structures having opposing wind directions, and the benefits associated therewith, as discussed herein with reference to FIG. 10.

In some implementations, the helical tube assembly can be positioned within a tube assembly of the present technology that further includes an intermediate member (such as intermediate member 120 and all variations discussed herein) with three steerable regions (such as steerable region 102), each axially coinciding with one of the helical structures. More or fewer steerable regions are possible. The intermediate member may coaxially surround the helical tube assembly, or the helical tube assembly can coaxially surround the intermediate member. Such tube assemblies may optionally include one or more additional elongate tubular members, such as outer and/or inner members as discussed above, and/or others. In some embodiments, the intermediate member and/or one or more other elongate tubular members may be disposed radially between the first and second elongate members 360, 380. In any case, the steering tendons (such as steering tendon 126) of the tube assembly may extend linearly through the respective steering regions while the strands of the axially coinciding helical structures extend helically. In any of the foregoing embodiments, incorporation of the first and second elongate members 360, 380 may reduce and/or prevent path length compensation issues that may otherwise arise in other tubular layers, as discussed in greater detail above.

In some cases, one or both of the first and second elongate members 360, 380 may have a length shorter than that of one, some, or all of the other elongate members of the tube assembly, including the intermediate member. In such embodiments, one or both of the first and second elongate members 360, 380 may not extend proximally to the proximal portion of the tube assembly and instead may terminate proximally along the intermediate portion 100c of the tube assembly. In other embodiments, one or both of the first and second elongate members 360, 380 have proximal portions that coincide with the proximal portion 100a of the tube assembly.

The helical tube assembly may also be used with tube assemblies without steering capabilities and can be aligned with passively flexible regions of one or more tubular layers of such assemblies.

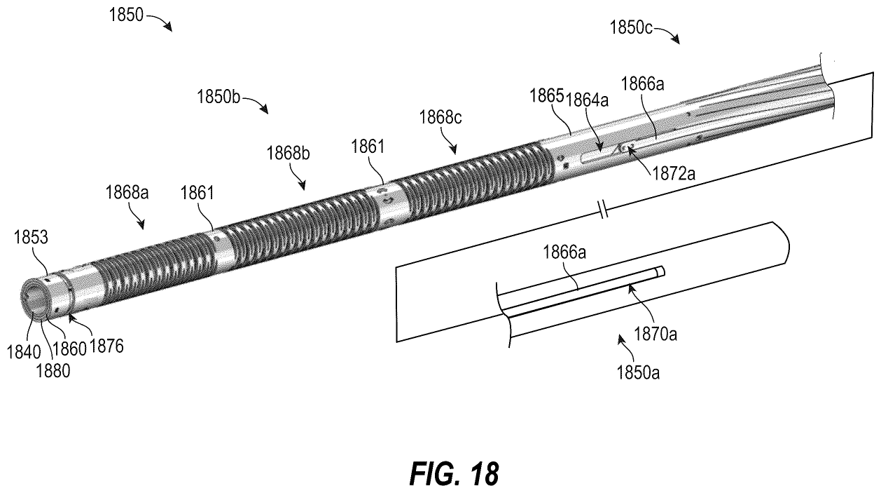

In some implementations, the helical tube assembly can be used within a tube assembly in which at least one of the elongate tubular members of the assembly, or at least a portion thereof, is configured to rotate relative to other elongate tubular members of the assembly. In such embodiments, the helical tube assembly may be fixed (e.g., via one or more laser welds) to the rotatable tube and/or rotatable portion of the tube such that the helical tube assembly is configured to rotate with the tube. An example mechanism is shown in FIGS. 18-26. FIG. 18 shows an example of a rotatable tube assembly 1850 (also referred to as “tube assembly 1850”) comprising a first elongate tubular member 1860 (“first member 1860”), a second elongate tubular member 1880 (“second member 1880”), and an optional third elongate tubular member 1840 (“third member 1840”), all positioned coaxially relative to one another. While the first member 1860 is shown positioned radially outwardly of the second member 1880, in some embodiments the second member 1880 may be positioned radially outwardly of the first member 1860. Several of the features of the rotatable tube assembly 1850 and first and second members 1860, 1880 can be generally similar to the features discussed above with reference to the tube assembly 100 and inner, intermediate, and outer members 110, 120, 130. For example, the tube assembly 1850 can have a proximal portion 1850a configured to be manipulated by a user, a distal portion 1850b configured to be disposed in the body lumen, and a passively flexible intermediate portion 1850c. Each of the first and second members 1860, 1880 can have distinct flexible regions 1868a-1868c, 1888a-1888c, respectively, separated by rigid portions 1861, 1881, respectively, of the sidewall. While three flexible regions are shown in the example of FIG. 18, in other embodiments the first and second members 1860, 1880 may have more or fewer flexible regions (e.g., no flexible regions, one flexible region, two flexible regions, four flexible regions, etc.). Unlike tube assembly 100, the first and second members 1860, 1880 do not have steering tendons. Instead, the flexible regions 1868a-1868c, 1888a-1888c remain either passively flexible (e.g., if the tube assembly 1850 is not incorporated into a steerable device) or are guided by a steerable elongate tubular member (such as intermediate member 120) incorporated into the tube assembly 1850. In the latter embodiments, the steerable tube assembly may be disposed radially outwardly or radially inwardly of the tube assembly 1850. In some embodiments, the first and/or second member 1860, 1880 may include steering tendons.

FIG. 19 is a side view of a distal region of the tube assembly 1850. As shown, the distal portion 1860b of the first member 1860 can include the flexible regions 1868a, 1868b, 1868c as well as a rigid portion 1865 just proximal of the proximalmost flexible region 1868c. Between the rigid portion 1865 and the proximal portion 1860a lies the passively flexible intermediate portion 1860c (only a distalmost region shown). The first member 1860 can further include diametrically opposed actuation tendons 1866a, 1866b (only 1866a visible in FIG. 19) slidably disposed in corresponding longitudinal slots 1864a, 1864b (only 1864a visible in FIG. 19). Both the actuation tendons 1866a, 1866b and the slots 1864a, 1864b may be cut from the tubular sidewall of the first member 1860 via the methods discussed herein. Each of the actuation tendons 1866a, 1866b have a proximal end portion 1870a, 1870b (only 1870a visible in FIG. 18) at the proximal portion 1850a of the tube assembly 1850 and a distal end portion 1872a, 1872b at the rigid portion 1865. The actuation tendons 1866a, 1866b may extend substantially linearly along the first member 1860, and/or may wrap helically around the longitudinal axis of the first member 1860. The proximal end portions 1870a, 1870b are configured to be directly or indirectly coupled to a steering interface, such as a handle or an interface for a robotic system.

FIG. 20 shows a distal region of the tube assembly 1850 with the first member 1860 removed for ease of viewing the second member 1880. As shown, the distal portion 1880b of the second member 1880 can include, from distal to proximal, a rigid distal end portion 1892, flexible regions 1888a, 1888b, 1888c, and a rotary actuator 1885, all formed of the tubular sidewall. Between the rotary actuator 1885 and the proximal portion 1860a lies the passively flexible intermediate portion 1880c (only a distalmost region shown). The rigid portion 1865 of the first member 1860 can be fixed (e.g., via welding, as discussed herein) at one or more locations to a distal end portion of the intermediate portion 1880c, as indicated by the dashed line. The rotary actuator 1885 is separated from the intermediate portion 1880c by a 360-degree cut 1894 in the sidewall such that the portion of the second member 1880 distal of the cut 1894, referred to herein as the “rotatable portion 1890,” is free to rotate independently of the portion of the sidewall proximal of the cut 1894, which includes the intermediate and proximal portions 1880c, 1880a. The rotatable portion 1890 thus includes the rotary actuator 1885, flexible regions 1888a-1888c, and distal end portion 1892.

As shown in FIG. 20, the rotary actuator 1885 can be partially separated from the proximalmost flexible region 1888c by a partial or fully circumferential slot 1887 in the sidewall. In some embodiments, the circumferential slot 1887 extends less than 360 degrees around the sidewall such that one or more bridge portions 1883 of the sidewall extends axially between the rotary actuator 1885 and the portion of the sidewall distal of the rotary actuator 1885. In other embodiments, the slot 1887 extends the full circumference uninterrupted (i.e., no bridge portion 1883). As shown in FIG. 19, the first member 1860 can include one or more lips 1867 disposed in a slot 1869 in the sidewall and that, when the tube assembly 1850 is fully assembled, extend radially inwardly (or outwardly if the first member 1860 is disposed outside of the second member 1880), into the circumferential slot 1887. The lip 1867 is shown in FIG. 19 immediately after the first member 1860 has been laser cut, and before being bent radially away from the first member sidewall during a subsequent manufacturing step. In some embodiments, the slot 1887 is in receipt of a lip from an elongate tubular member on the opposite side of the second member 1880 as the first member 1860, which may be in addition to or instead of the lip(s) 1867 of the first member 1860. In either case, the lip 1867 prevents axial movement of the rotatable portion 1890 while allowing relative rotation of the rotatable portion 1890.

The second member 1880 can further include first and second helical slots 1884a, 1884b and first and second sliders 1886a, 1886b slidably disposed in a corresponding one of the slots 1884a, 1884b (only 1886a visible in FIG. 20). Both the first and second slots 1884a, 1884b and first and second sliders 1886a, 1886b may be cut from the tubular sidewall of the second member 1880, in particular along the portion of the sidewall forming the rotary actuator 1885. When the tube assembly 1850 is fully assembled, the rigid portion 1865 of the first member 1860 is axially aligned with the rotary actuator 1885 and the distal end portions 1872a, 1872b of the actuation tendons 1866a, 1866b are aligned with and fixed to a corresponding slider 1886a, 1886b (e.g., via welding, as discussed herein). The actuation tendons 1866a, 1866b are blocked from tangential movement by the adjacent portions of the first member sidewall, and thus the attached sliders 1886a, 1886b also cannot move tangentially. As such, axial movement of the actuation tendons 1866a, 1866b causes axial movement of the sliders 1886a, 1886b, which because of the helical slots 1884a, 1884b causes rotation of the rotary actuator 1885 and thus the rotatable portion 1890. Additionally, the axial forces created by actuation of the actuation tendons 1866a, 1866b are constrained by the engagement between the lip 1867 and slot 1887.

In some embodiments, the distal end portion 1892 of the second member 1880 is fixed at one or more locations (e.g., via welding, as discussed herein) to a rigid portion 1853 at the distal end of the first member 1860. The rigid portion 1853 may comprise a portion of the tubular sidewall of the second member 1880 that is separated from the flexible regions 1868a-1868c by a fully-circumferential (e.g., 360 degree) slot 1876. The slot 1876 beneficially allows for some compression of the second member 1880 when axial forces are high. Moreover, the slot 1876 allows the rigid portion 1853 to rotate with the rotatable portion 1890.

When the rotatable portion 1890 rotates, the first member 1860 (except for the rigid portion 1853) remains rotationally fixed (e.g., does not rotate relative to other elongate tubular members of the tube assembly) as does the portion of the second member 1880 proximal of the rotatable portion 1890. When the tube assembly 1850 is integrated with a steerable tube assembly, the elongate tubular members of the steerable tube assembly also remain rotationally fixed. As such, the elongate tubular member having the steering tendons does not rotate and any deflected orientation of the steerable region(s) remains the same when the rotatable portion 1890 rotates. The present technology thereby enables rotation of a portion of one elongate tubular member relative to the other elongate tubular members of the assembly while the assembly is in a curved configuration. The non-rotating elongate members can thus maintain stable positioning in the anatomy while the rotating elongate member is manipulated to modify tissue, image an area of interest, etc. In this way, a working tool or end effector attached to the distal tip of rotatable portion 1890, can be rotated from the proximal end by operating (axially pushing or pulling) actuation tendons 1866a, 1866b to provide that working tool or end effector with a desired orientation while the tube assembly itself—apart from the rotatable portion 1890—does not rotate.

The tube assembly 1850 may optionally include one or more elongate tubular members disposed at a radial side of the second member 1880 opposite that of the first member 1860. The third member 1840 (FIG. 18), for example, may comprise one or more elongate tubular members. FIG. 18 shows the third member 1840 disposed radially inwardly of (e.g., in the lumen of) the second member 1860. In other embodiments, the first member 1860 can be radially inward of (e.g., in the lumen of) the second member 1880 and the second member 1880 can be radially inward of (e.g., in the lumen of) the third member 1840. In some examples, the third member 1840 includes one or more support members that are fixed at one or more locations (e.g., via welding, as discussed herein) to the second member 1880, and to one another (if more than one support member is used). The support member(s) can comprise an elongate tubular sidewall having flexible regions coinciding with the flexible regions of the second member 1880. The support member(s) beneficially adds axial and torsional stiffness to the second member 1880 while maintaining flexibility.

In certain examples, the support member comprises one or more helical structures, such as the helical structures shown in FIGS. 16A-17B. For example, some embodiments of the present technology include a tube assembly comprising at the least one or both of the of the first and second elongate tubes 360, 380, as well as the first and second elongate tubular members 1860, 1880. The first and/or second elongate tubes 360, 380 can be positioned coaxially relative to the first and second elongate tubular members 1860, 1880 such that the first helical structures 362a, 372a can be axially aligned with all or a portion of the first flexible regions 1868a, 1888a, the second helical structures 362b, 372b can be axially aligned with all or a portion of the second flexible regions 1868b, 1888b, and/or the third helical structures 362c, 372c can be axially aligned with all or a portion of the third flexible regions 1868c, 1888c. The number of helical structures per elongate tubular member can depend on the number of flexible regions. The first and/or second elongate tubes 360, 380 can be fixed to the first and second elongate tubular members 1860, 1880, for example via welding at one or more locations along the rigid bands on either side of and between the helical structures and flexible regions.

In any case, the support member(s) can have a length shorter than the second member 1880 such that it extends along only a distal portion of the tube assembly 1850. In other embodiments, the support member(s) extend proximally to the proximal portion of the tube assembly 1850.

The third elongate member 1840 can thus be 1) one or more elongate tubular members disposed immediately adjacent the second member 1880 but not fixed to the rotatable portion 1890 (and thus not rotatable with the rotatable portion 1890), 2) one or more support members immediately adjacent the second member 1880 and fixed to and rotatable with at least the rotatable portion 1890), or 3) one or more support members immediately adjacent the second member 1880 and fixed to and rotatable with at least the rotatable portion 1890 and one or more other elongate tubular members not fixed to the rotatable portion 1890, where the support member(s) are positioned between the other elongate tubular members and the second member 1880.

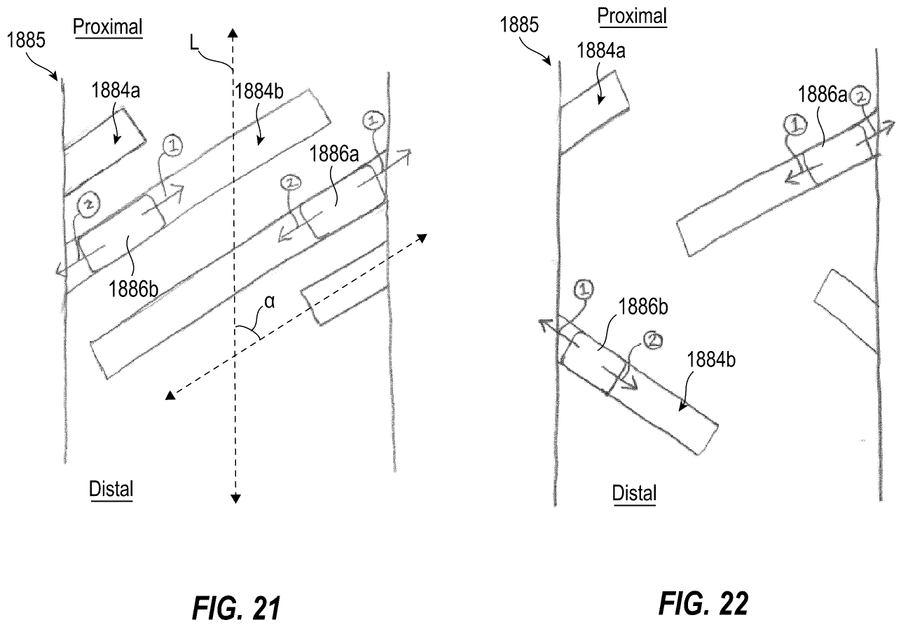

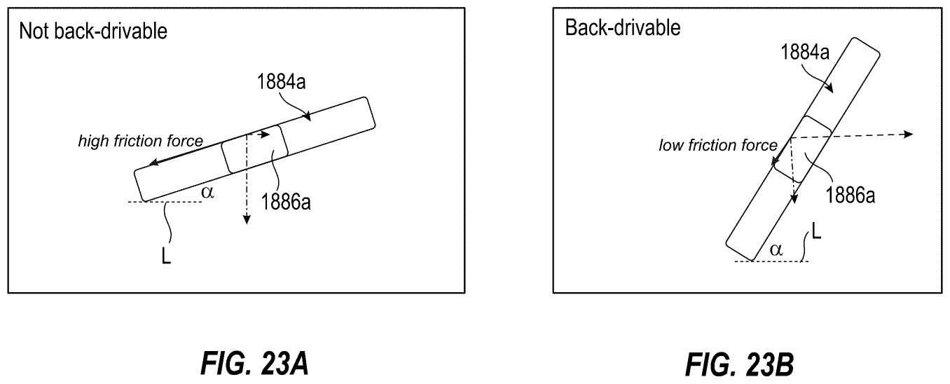

As shown in FIG. 20 and the laid flat view of the rotary actuator 1885 in FIG. 21, in some embodiments the first and second helical slots 1884a, 1884b wrap around the longitudinal axis L in the same direction (both clockwise or both counterclockwise). Each of the first and second slots 1884a, 1884b can be angled with respect to the longitudinal axis L by an angle α, which may be no less than 5 degrees, no less than 10 degrees, no less than 20 degrees, or no less than 30 degrees, from about 20 degrees to about 50 degrees, from about 30 degrees to about 40 degrees, about 30 degrees, about 35 degrees, or about 40 degrees. The angle α may be selected based on a desired back-drivability of the actuation tendons, as discussed in greater detail below with reference to FIGS. 23A and 23B. In the example slot arrangement of FIG. 21, pushing both actuation tendons 1866a, 1866b distally causes the sliders 1886a, 1886b to move distally (arrow 2) such that the rotary actuator 1885 (and thus rotatable portion 1890) rotate in a first direction. Pulling both actuation tendons 1866a, 1866b proximally causes the sliders 1886a, 1886b to move proximally (arrow 1) such that the rotary actuator 1885 (and thus the rotatable portion 1890) rotates in a second direction opposite the first direction. It will be appreciated that the tube assembly 1850 can be configured with more or fewer than two actuation tendons and corresponding helical slots and sliders, such as a single actuation tendon, helical slot, and slider, or three or more actuation tendons, helical slots, and sliders. Moreover, in those embodiments in which multiple actuation tendons are used, the spacing between the tendons may be evenly distributed about the circumference (e.g., 180 degrees between two tendons, 120 degrees between three tendons, 90 degrees between four tendons, etc.) or may be unevenly distributed.

As shown in the laid flat view of the rotary actuator 1885 in FIG. 22, in some embodiments the first and second helical slots 1884a, 1884b wrap around the longitudinal axis L in opposite directions (one clockwise and the other counterclockwise). The first slot 1884a can be angled with respect to the longitudinal axis L by an angle α, while the second slot 1884b can be angled with respect to the longitudinal axis L by (−α). The angle α may be no less than 5 degrees, no less than 10 degrees, no less than 20 degrees, or no less than 30 degrees, from about 20 degrees to about 50 degrees, from about 30 degrees to about 40 degrees, about 30 degrees, about 35 degrees, or about 40 degrees. The angle α may be selected based on a desired back-drivability of the actuation tendons, as discussed in greater detail below with reference to FIGS. 23A and 23B. In the example slot arrangement of FIG. 22, pulling the first actuation tendon 1866a proximally while pushing the second actuation tendon 1866b distally causes the rotary actuator 1885 (and thus rotatable portion 1890) to rotate in a first direction, while pushing the first actuation tendon 1866a distally while pulling the second actuation tendon 1866b proximally causes the rotary actuator 1885 (and thus rotatable portion 1890) to rotate in a second direction opposite the first direction. It will be appreciated that the tube assembly 1850 can be configured with any multiple of two actuation tendons and corresponding helical slots and sliders, such as four actuation tendons, helical slots, and sliders, six actuation tendons, helical slots, and sliders, etc. Moreover, the spacing between the tendons may be evenly distributed about the circumference (e.g., 180 degrees between two tendons, 120 degrees between three tendons, 90 degrees between four tendons, etc.) or may be unevenly distributed.

As previously mentioned, the angle α of the helical slots can be varied to control the back-drivability of the actuation tendons. In some cases it may be desirable to prevent forces exerted at the distal tip of the tube assembly from back-driving the actuation tendons, which can lead to unwanted rotation of the rotatable portion 1890. The lower the angle α of the slot 1884a with respect to the longitudinal axis L of the tube assembly 1850, the higher the rotational force needed to back-drive the tendons, as shown in FIGS. 23A and 23B, and the higher the gear reduction ratio (i.e., the amount of axial displacement of the tendon required to rotate the rotatable portion 1890). In other applications it may be desirable to have back-drivability (e.g., allow the tip to rotate when there are forces present), for example for the user or system to have a better sense/feel for such forces. In these cases, the angle α of the slot 1884a may be higher.

In some embodiments, an end effector may be operatively coupled to the distal end of the second member 1880 such that rotation of the rotatable portion 1890 causes rotation of the end effector 2400. The rotatable tube assemblies disclosed herein can be used with a variety of end effectors depending on the intended medical application. In some embodiments, the end effector may comprise an imaging device such as an optical coherence tomography (OCT) probe, an ultrasound transducer, or a fiber optic camera to provide visualization of the treatment area. In some implementations, the end effector may comprise a therapeutic device such as a laser fiber for ablation procedures, a radiofrequency electrode for tissue modification, a cryotherapy probe for freezing tissue, or an ultrasound energy emitter for therapeutic applications. The end effector may comprise a mechanical tool such as a grasper for tissue manipulation, a cutting blade or scissors for tissue dissection, a needle for injection or aspiration, a snare for capturing and removing foreign objects or tissue, or a basket for stone retrieval. In some implementations, the end effector may comprise a deployment mechanism for delivering implants such as stents, coils, embolic devices, or drug-eluting devices. The end effector may be a clip applier. The end effector may comprise a suction port for aspiration of fluids or emboli, or a fluid delivery port for irrigation or drug delivery. In some implementations, the end effector may comprise sensors for measuring physiological parameters such as pressure, temperature, pH, or oxygen saturation. The end effector may also comprise a combination of multiple functional elements, such as an imaging device integrated with a therapeutic element, to provide simultaneous visualization and treatment capabilities.

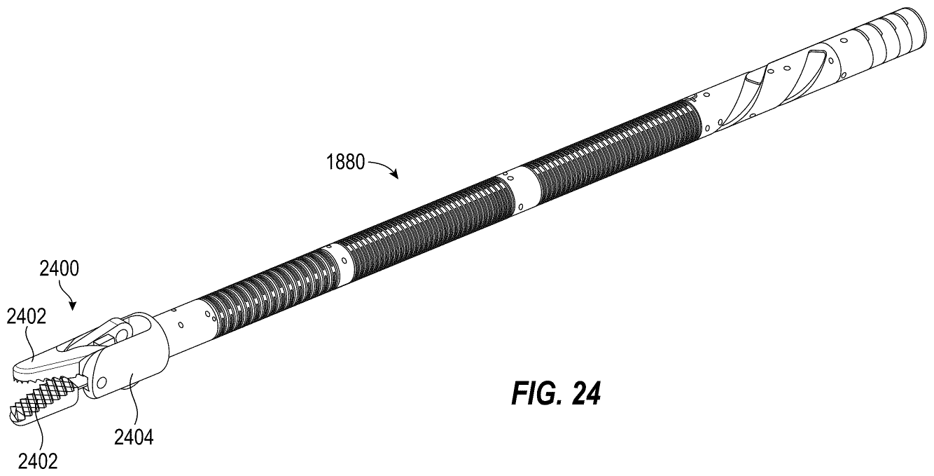

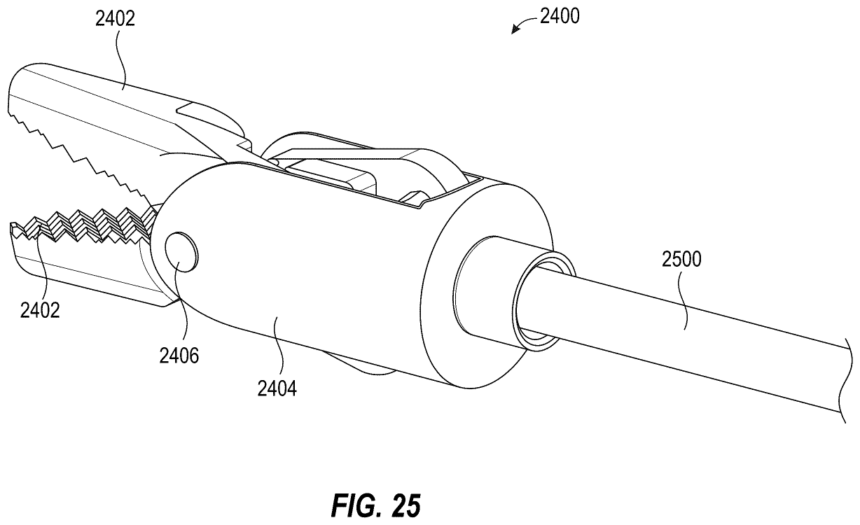

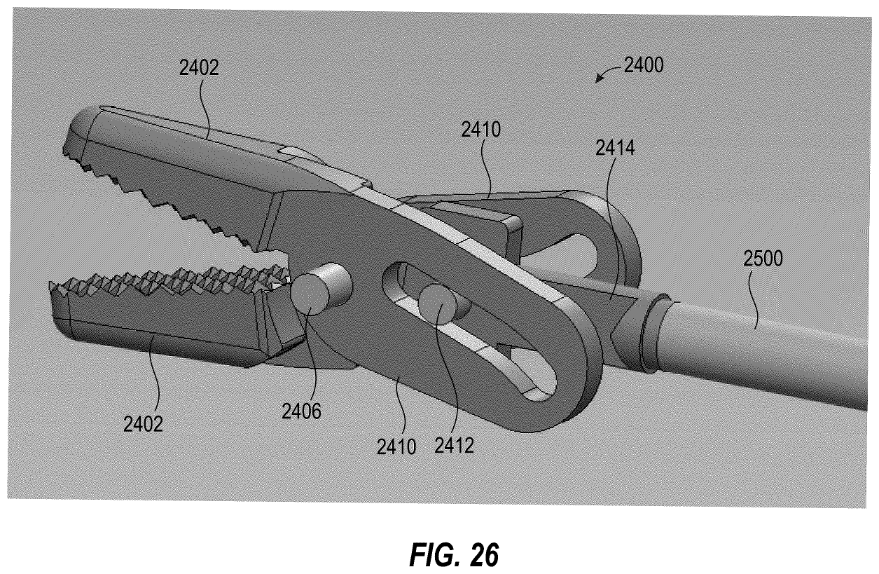

An example end effector 2400 for use with the tube assembly 1850 is illustrated in FIG. 24, shown attached to the distal end of the second member 1880. FIG. 25 shows the same configuration of FIG. 24 but with the second member 1880 removed, and FIG. 26 shows the same configuration of FIG. 25 but with the clevis 2404 removed. Referring to FIGS. 24-26 together, the end effector 2400 can include first and second jaws 2402 rotatably mounted on a pin 2406 secured to a clevis 2404. The clevis 2404 can be fixed to the distal end of the rotatable portion 1890 such that rotation of the rotatable portion 1890 causes rotation of the clevis 2404 (and thus the end effector). Each jaw 2402 has a slot in receipt of another pin 2412 fixed to a drive plate 2414. A proximal end of the drive plate 2414 is fixed to a control member 2500 that extends through a lumen of the tube assembly 1850, such as through a lumen of the second or third member 1880, 1840. In some examples, the control member 2500 comprises an elongate tube, rod, cable, etc. which is axially stiff but flexible to accommodate passive bending of the intermediate portion 1850c of the assembly 1850. As best visualized in FIG. 26, axial movement of the control member 2500 causes the jaws 2402 to rotate around the pin 2406, thereby causing the jaws 2402 to open and close.

According to some embodiments, the rotary actuator 1885 can be located distal of the flexible regions or may be disposed at any location between them. However, in such embodiments, bending of the flexible region(s) proximal of the rotary actuator 1885 may cause path length changes of the actuation tendon(s), as the actuation tendon would extend through any flexible region proximal of the rotary actuator 1885 to reach the rotary actuator 1885. In such embodiments, the tube assembly may include one or more mechanisms for compensating path length changes for the actuation tendon(s). Moreover, bending over a large angle one or more flexible regions located proximal of the rotary actuator 1885 may cause undesired strain in the portion of the actuation tendon inside the bent flexible region.

Some methods of making a rotatable tube assembly include providing the first member 1860, providing the second member 1880, and inserting the first member 1860 within a lumen of the second member 1880, or vice versa. The method can further include bending the lip 1867 of the first member 1860 radially away from the sidewall of the first member 1860 into the slot 1887 in the sidewall of the second member 1880. The method can further include fixing the distal end portion of the actuation tendons 1866a, 1866b to the sliders 1886a, 1886b of the rotary actuator 1885 such that axial movement of the actuation tendons 1866a, 1866b causes rotation of the rotatable portion 1890 around a longitudinal axis extending through the distal portion. In some embodiments, the method further includes positioning a third or more elongate members coaxially relative to the first and second elongate members. In some examples, the method includes cutting the rotary actuator from the tubular sidewall of the second elongate member along the rotatable portion.

Any of the foregoing embodiments can optionally include a polymer jacket and/or inner liner. For example, such a jacket or liner could be arranged inside all or a portion of helical structure 300, inside all or a portion of helical structure 321, inside all or a portion of first elongate member 360, inside all or a portion of second elongate member 380, between all or a portion of helical structures 300, 321, between all or a portion of first and second elongate members 360, 380, outside all or a portion of helical structure 300, outside all or a portion of helical structure 321, outside all or a portion of first elongate member 360, or outside all or a portion of second elongate member 380. Such a jacket or liner can be applied on an exterior surface of the respective helical structure or member such that the polymer extends radially between some or all of the strands and thus provides radially extending spacers between the strands.

In some embodiments, radially adjacent coaxial elongate tubular members, including the first, second, and third elongate members 360, 380, 396 and others, may have a radial play in a range of about 0.01 mm to about 0.3 mm.

### EXAMPLES

The subject technology is illustrated, for example, according to various aspects described below, including with reference to FIGS. 1-26. Various examples of aspects of the subject technology are described as numbered examples (1, 2, 3, etc.) for convenience. These are provided as examples and do not limit the subject technology.

Example 1: An invasive instrument, e.g. for medical applications, extending in an axial direction and having at least one deflectable zone and comprising a first tube and at least one steering wire configured to deflect the deflectable zone by an axial movement, the first tube having a first helical structure located at the at least one deflectable zone, the first helical structure comprising a first plurality of helically arranged first strands with a first lead length in which the first plurality of helically arranged first strands are circling around 360 degrees in a non-deflected state of the at least one deflectable zone, the at least one deflectable zone having a deflectable length of n1 times the first lead length, n1 being an integer value >=1, the invasive instrument being configured such that it is deflectable in the at least one deflectable zone and rigid in at least portions at both axial sides of the deflectable zone, the first plurality of helically arranged first strands resulting from a material removal technique applied to the first tube.

Example 2: The invasive instrument according to Example 1, wherein the first plurality of helically arranged first strands of the first helical structure extend beyond the at least one deflectable zone.

Example 3: The invasive instrument according to Example 1 or 2, having at least one of the following features: (a) the first plurality of helically arranged first strands comprise four or more helically arranged first strands, (b) the four or more helically arranged first strands are arranged equidistantly, (c) the four or more helically arranged first strands have a thickness in a range of 0.03-2.0 mm, 0.03-1.0 mm, 0.05-0.5 mm, or 0.08-0.4 mm, (d) the four or more helically arranged first strands have a width in a range of 0.005-2 mm, (e) the four or more helically arranged first strands have a uniform width along their entire length, (f) the four or more helically arranged first strands are separated by first slots having a first slot width in a range of 0.01-3 mm, (g) the first tube length is in a range of 5-100 mm, and/or (h) the first tube has a first tube diameter in a range of 1-20 mm.

Example 4: The invasive instrument according to Example 3, wherein all strands of the helically arranged first strands have at least one of an equal first length, an equal first width or an equal first thickness.

Example 5: The invasive instrument according to any of the preceding Examples, wherein the helically arranged strands are arranged equidistantly.

Example 6: The invasive instrument according to any of the preceding Examples, wherein the invasive instrument comprises a second tube coaxially arranged with the first tube and having a second helical structure located at the at least one deflectable zone, the second helical structure comprising a second plurality of helically arranged second strands with a second lead length in which the second plurality of helically arranged second strands are circling around 360 degrees in a non-deflected state of the at least one deflectable zone, the second plurality of helically arranged second strands circling n2 times the second lead length in the deflectable zone, where n2 is an integer value >=1, the second plurality of helically arranged second strands resulting from a material removal technique applied to the second tube, the first strands circling in a first direction and the second strands circling in a second, opposite direction.

Example 7: The invasive instrument according to Example 6, wherein the first lead length is equal to the second lead length and n1=n2.

Example 8: The invasive instrument according to Example 6 or 7, wherein the second plurality of helically arranged strands of the second helical structure extend beyond the at least one deflectable zone.

Example 9: The invasive instrument according to Example 6, 7 or 8, having at least one of the following features: (a) the second plurality of helically arranged strands comprise four or more helically arranged second strands, (b) the four or more helically arranged second strands are arranged equidistantly, (c) the four or more helically arranged second strands have a thickness in a range of 0.03-2.0 mm, preferably 0.03-1.0 mm, more preferably 0.05-0.5 mm, and most preferably 0.08-0.4 mm, (d) the four or more helically arranged second strands have a width in a range of 0.005-2 mm, (e) the four or more helically arranged second strands have a uniform width along their entire length, (f) the four or more helically arranged second strands are separated by second slots having a second slot width in a range of 0.01-3 mm, (h) the second tube length is in a range of 5-100 mm, and/or (i) the second tube has a second tube diameter in a range of 1-20 mm.

Example 10: The invasive instrument according to any of the Examples 6 to 9 in their dependency on Example 4, wherein all strands of the second plurality of strands have at least one of an equal second length, an equal second width and an equal second thickness, and the second length, second width and second thickness, respectively, may be equal to the first length, first width and first thickness, respectively.

Example 11: The invasive instrument according to any of the preceding Examples, the invasive instrument having at least one of (a) a third tube with a first flexible hinge structure, the third tube being coaxially arranged outside the first tube, the first flexible hinge structure extending along a third tube length equal to the deflectable length and axially aligned with the first strands, the third tube being rigid in at least portions at both axial sides of the third tube length, or (b) a fourth tube with a second flexible hinge structure, the fourth tube being coaxially arranged inside the first tube, the second flexible hinge structure extending along a fourth tube length equal to the deflectable length and axially aligned with the first strands, the fourth tube being rigid in at least portions at both axial sides of the fourth tube length.

Example 12: The invasive instrument according to any of the Examples 6 to 10, wherein the second tube surrounds the first tube and the invasive instrument has at least one of (a) a third tube with a first flexible hinge structure, the third tube being coaxially arranged outside the second tube, the first flexible hinge structure extending along a third tube length equal to the deflectable length and axially aligned with the second strands, the third tube being rigid in at least portions at both axial sides of the third tube length, or (b) a fourth tube with a second flexible hinge structure, the fourth tube being coaxially arranged inside the first tube, the second flexible hinge structure extending along a fourth tube length equal to the deflectable length and axially aligned with the first strands, the fourth tube being rigid in at least portions at both axial sides of the fourth tube length.

Example 13: The invasive instrument according to any of the Examples 6 to 12, wherein adjacent coaxial tubes have a radial play in a range of 0.01-0.3 mm.

Example 14: An invasive instrument, e.g. for medical applications, extending in an axial direction and having at least one deflectable zone and comprising a first tube and at least one steering wire configured to deflect the deflectable zone by an axial movement, the first tube having a first helical structure located at a flexible zone, the first helical structure comprising a first plurality of helically arranged first strands with a first lead length in which the first plurality of helically arranged first strands are circling around 360 degrees in a non-bent state of the flexible zone, the flexible zone having a flexible length of n1 times the first lead length, n1 being an integer value >=1, the invasive instrument being configured such that it is flexible in the flexible zone and rigid in at least portions at both axial sides of the flexible zone, the first plurality of helically arranged first strands resulting from a material removal technique applied to the first tube, the invasive instrument comprising a second tube coaxially arranged with the first tube and having a second helical structure located at the flexible zone, the second helical structure comprising a second plurality of helically arranged second strands with a second lead length in which the second plurality of helically arranged second strands are circling around 360 degrees in a non-deflected state of the flexible zone, the second plurality of helically arranged second strands circling n2 times the second lead length in the flexible zone, where n2 is an integer value >=1, the second plurality of helically arranged second strands resulting from a material removal technique applied to the second tube, the first strands circling in a first direction and the second strands circling in a second, opposite direction.

Example 15: The invasive instrument according to Example 14 or 15, having at least one of the following features: (a) the first plurality of helically arranged strands of the first helical structure extend beyond the flexible zone, or (b) the second plurality of helically arranged strands of the second helical structure extend beyond the flexible zone.

Example 16: The invasive instrument according to Example 14 or 15, wherein all strands of the helically arranged first strands (302) have at least one of an equal first length, an equal first width or an equal first thickness and all strands of the second plurality of second strands (322) have at least one of an equal second length, an equal second width and an equal second thickness, and the second length, second width and second thickness, respectively, may be equal to the first length, first width and first thickness, respectively.

Example 17: The invasive instrument according to any of the Examples 14 to 17, wherein the second tube surrounds the first tube and the invasive instrument has at least one of: (a) a third tube with a first flexible hinge structure, the third tube being coaxially arranged outside the second tube, the first flexible hinge structure extending along a third tube length equal to the deflectable length and axially aligned with the second strands, the third tube being rigid in at least portions at both axial sides of the third tube length, or a fourth tube with a second flexible hinge structure, the fourth tube being coaxially arranged inside the first tube, the second flexible hinge structure extending along a fourth tube length equal to the deflectable length and axially aligned with the first strands, the fourth tube being rigid in at least portions at both axial sides of the fourth tube length.

Example 18: An instrument comprising: an elongate member having a proximal portion, a distal portion and an intermediate portion extending in a longitudinal direction between the proximal and distal portions, wherein the elongate member further includes: a first elongate tube comprising a sidewall having a first proximal band, a first distal band, and a first flexible region extending axially between the first proximal and distal bands, wherein the first flexible region comprises a plurality of first strands extending helically around a longitudinal axis of the first elongate tube in a first circumferential direction between the first proximal and distal bands, and a second elongate tube coaxial with the first elongate tube, wherein the second elongate tube comprises a sidewall having a second proximal band, a second distal band, and a second flexible region extending axially between the second proximal and distal bands, wherein the second flexible region comprises a plurality of second strands extending helically around a longitudinal axis of the second elongate tube in a second circumferential direction between the second proximal and distal bands, the second circumferential direction opposite the first circumferential direction, wherein the first and second flexible regions are at least partially axially aligned with one another.

Example 19: The instrument of Example 18, wherein the first and second proximal bands are aligned and fixed to one another and the first and second distal bands are aligned and fixed to one another.

Example 20: The instrument of Example 18 or Example 19, wherein the elongate member further comprises a third elongate tube coaxial with the first and second elongate tubes.

Example 21: The instrument of Example 20, wherein the elongate member further comprises a fourth elongate tube coaxial with the first, second, and third elongate tubes.

Example 22: The instrument of any one of Examples 18 to 20, wherein the first strands extend helically around the first elongate tube at least 360 degrees between the first proximal and distal bands.

Example 23: The instrument of any one of Examples 18 to 22, wherein the second strands extend around the second elongate tube at least 360 degrees between the second proximal and distal bands.

Example 24: The instrument of any one of Examples 18 to 23, wherein the first strands extend around 360 degrees of the first elongate tube N times between the first proximal and distal bands, wherein N is an integer of 1 or more.

Example 25: The instrument of any one of Examples 18 to 24, wherein the second strands extend around 360 degrees of the second elongate tube N times between the second proximal and distal bands, wherein N is an integer of 1 or more.

Example 26: The instrument of any one of Examples 18 to 25, wherein: the first helical strands extend around 360 degrees of the first elongate tube N times between the first proximal and distal bands, wherein N is an integer of 1 or more, and wherein the second helical strands extend around 360 degrees of the second elongate tube N times between the second proximal and distal bands.

Example 27: The instrument of any one of Examples 18 to 26, wherein: the first helical strands extend around 360 degrees of the first elongate tube N times between the first proximal and distal bands, wherein N is an integer of 1 or more, and wherein the second helical strands extend around the second elongate tube more than an integer multiple of 360 degrees between the second proximal and distal bands.

Example 28: An instrument comprising: an elongate member having a proximal portion, a distal portion, an actuation region at the proximal portion, an articulable region at the distal portion, and: a first elongate tube comprising a first sidewall and having a first flexible region along which the first elongate tube is configured to bend, and a second elongate tube coaxial with the first elongate tube, wherein the second elongate tube comprises a second sidewall having a second flexible region comprising a plurality of helical strands formed of the second sidewall, and wherein the second flexible region is at least partially axially aligned with the first flexible region.

Example 29: The instrument of Example 28, wherein the second flexible region is disposed between a first rigid band and a second rigid band, each of the first and second rigid bands formed of the second sidewall.

Example 30: The instrument of Example 29, wherein the strands extend around the second elongate tube at least 360 degrees between the first and second rigid bands.

Example 31: The instrument of Example 29, wherein the strands extend around 360 degrees of the second elongate tube N times between the first and second bands, wherein N is an integer of 1 or more.

Example 32: The instrument of any one of Examples 28 to 31, wherein the first flexible region is disposed between a first rigid band and a second rigid band, each of the first and second rigid bands formed of the first sidewall.

Example 33: The instrument of Example 32, wherein the first bands of the first and second sidewalls are axially aligned and fixed to one another and the second bands of the first and second sidewalls are axially aligned and fixed to one another.

Example 34: The instrument of any one of Examples 28 to 33, wherein the elongate member further comprises a third elongate tube coaxial with the first and second elongate tubes.

Example 35: The instrument of any one of Examples 28 to 34, wherein the elongate member further comprises a fourth elongate tube coaxial with the first, second, and third elongate tubes.

Example 36: An instrument comprising: an elongate member having a proximal portion, a distal portion, an actuation region at the proximal portion, an articulable region at the distal portion, and: a first elongate tube comprising a sidewall and having a tendon formed of the sidewall, the tendon extending between the actuation region and the articulable region and configured to transfer a force applied at the actuation region to the articulable region to cause articulation of the articulable region, and wherein the first elongate tube comprises a first flexible region axially coinciding with the articulable region and through which the tendon extends to control articulation, and a second elongate tube coaxial with the first elongate tube, wherein the second elongate tube comprises a sidewall having a second flexible region comprising a plurality of helical strands formed of the sidewall, and wherein the second flexible region is at least partially axially aligned with the first flexible region.

Example 37: The instrument of Example 36, wherein the first flexible region extends between a first distal band and a first proximal band, each formed of the sidewall of the first elongate tube, and wherein a distal end portion of the tendon is attached to the first distal band.

Example 38: The instrument of Example 37, wherein: the helical strands of the second flexible region extend between a second distal band and a second proximal band, each formed of the sidewall of the second elongate tube, and wherein the first and second distal bands are aligned with and fixed to one another and the first and second proximal bands are aligned with and fixed to one another.

Example 39: The instrument of any one of Examples 36 to 38, wherein the helical strands extend around 360 degrees of the second elongate tube N times along the length of the second flexible region, wherein N is an integer of 1 or more.

Example 40: The instrument of Example 39, further comprising a third elongate tube coaxial with the first and second elongate tubes.

Example 41: The instrument of Example 40, wherein the helical strands are first helical strands that extend helically around a longitudinal axis of the first elongate tube in a first circumferential direction and wherein: the third elongate tube comprises a sidewall having a third flexible region comprising a plurality of second helical strands formed of the sidewall and the third flexible region is axially aligned with the first and second flexible regions, and the second helical strands extend helically around a longitudinal axis of the third elongate tube in a second circumferential direction opposite the first circumferential direction.

Example 42: The instrument of any one of Examples 28 to 41, wherein the articulable region is configured to bend omnidirectionally.

Example 43: The instrument of any one of Examples 28 to 42, wherein the portion of the tendon extending through the articulating region is substantially linear.

Example 44: An instrument comprising: an elongate member having a proximal portion, a distal portion, an actuation region at the proximal portion, an articulable region at the distal portion, and: a first elongate tube comprising a sidewall and having a tendon formed of the sidewall, the tendon extending between the actuation region and the articulable region and configured to transfer a force applied at the actuation region to the articulable region to cause articulation of the articulable region, and wherein the first elongate tube comprises a first flexible region axially coinciding with the articulable region and through which the tendon extends to control articulation, a second elongate tube coaxial with the first elongate tube, wherein the second elongate tube comprises a sidewall having a second flexible region comprising a plurality of helical strands formed of the sidewall, and a third elongate tube coaxial with the first and second elongate tubes, wherein the third elongate tube comprises a sidewall having a third flexible region, and wherein the first, second, and third flexible regions are at least partially axially aligned.

Example 45: The instrument of Example 44, wherein the helical strands extend around the second elongate tube at least 360 degrees along the second flexible region.

Example 46: The instrument of Example 44 or 45, wherein each of the first, second, and third flexible regions are axially bound by proximal and distal bands formed of the respective first, second, or third elongate tube, and wherein the proximal bands of each of the first, second, and third elongate tubes are fixed to one another and the distal bands of each of the first, second, and third elongate tubes are fixed to one another.

Example 47: The instrument of any one of Examples 44 to 46, wherein the helical strands are separated by slots comprising helically extending openings in the sidewall of the second elongate tube.

Example 48: The instrument of any one of Examples 44 to 47, wherein the articulable region is configured to bend omnidirectionally.

Example 49: An instrument comprising: an elongate member having a proximal portion, a distal portion, and a longitudinal axis extending therebetween, wherein the elongate member further includes: a first elongate tube comprising a sidewall having a first proximal band, a first distal band, and a first flexible region extending axially between the first proximal and distal bands, wherein the first flexible region comprises a plurality of first strands extending helically around a longitudinal axis of the first elongate tube in a first chirality between the first proximal and distal bands, and a second elongate tube coaxial with the first elongate tube, wherein the second elongate tube comprises a sidewall having a second proximal band, a second distal band, and a second flexible region extending axially between the second proximal and distal bands, wherein the second flexible region comprises a plurality of second strands extending helically around a longitudinal axis of the second elongate tube in a second chirality opposite the first chirality, wherein the first and second flexible regions are at least partially axially aligned with one another.

Example 50: An instrument comprising: an elongate member formed of a tubular sidewall, wherein at least a portion of the sidewall comprises a plurality of bridge regions and a plurality of struts regions alternating with the plurality of bridge regions along a circumference of the elongate member such that the strut regions extend circumferentially between and connect adjacent bridge regions, and wherein: each of the plurality of bridge regions comprise a plurality of bridges axially spaced apart from one another, each of the plurality of strut regions comprise a plurality of struts, each of the struts extending between bridges of circumferentially adjacent bridge regions, and at least when the at least a portion of the sidewall is in a straight configuration, each of the struts extend at a non-perpendicular angle relative to a longitudinal axis of the elongate member.

Example 51: The instrument of Example 50, wherein the at least a portion of the tubular sidewall is configured to bend omnidirectionally.

Example 52: The instrument of Example 50 or 51, wherein axially adjacent struts within a respective strut region extend towards opposite axial directions from the same bridge.

Example 53: The instrument of any one of Examples 50 to 52, wherein the elongate member includes at least two bridge regions and at least two strut regions.

Example 54: The instrument of any one of Examples 50 to 53, wherein the elongate member includes at least four bridge regions and at least four strut regions.

Example 55: The instrument of any one of Examples 50 to 54, wherein the bridges of circumferentially adjacent bridge regions are axially offset from one another.

Example 56: The instrument of any one of Examples 50 to 55, wherein at least some of the bridges surround and define an opening.

Example 57: The instrument of any one of Examples 50 to 56, wherein at least some of the bridges connect to four struts.

Example 58: The instrument of Example 57, wherein, for the at least some of the bridges: a first one of the connecting struts extends away from the respective bridge in a first circumferential direction and a first axial direction, a second one of the connecting struts extends away from the respective bridge in a first circumferential direction and a second axial direction opposite the first axial direction, a third one of the connecting struts extends away from the respective bridge in a second circumferential direction opposite the first circumferential direction and in the first axial direction, and a fourth one of the connecting struts extends away from the respective bridge in the second circumferential direction and the second axial direction.

Example 59: The instrument of any one of Examples 50 to 58, wherein the elongate member is a first elongate member and wherein the instrument further comprises a second elongate member coaxial with the first elongate member.

Example 60: The instrument of Example 59, wherein the tubular sidewall is a first tubular sidewall and the second elongate member comprises a second tubular sidewall having a flexible region, the flexible region comprising a plurality of strands formed of the second tubular sidewall and extending helically around a longitudinal axis of the second elongate member, and wherein the flexible region at least partially axially coincides with the at least a portion of the first tubular sidewall.

Example 61: An instrument comprising: an elongate member comprising: a first elongate tube comprising a flexible region; a second elongate tube coaxial with the first elongate tube and comprising a sidewall, wherein at least a portion of the sidewall axially coincides with the flexible region and comprises a plurality of bridge regions and a plurality of struts regions alternating with the plurality of bridge regions along a circumference of the second elongate tube such that the strut regions extend circumferentially between and connect the bridge regions, and wherein: each of the plurality of bridge regions comprise a plurality of bridges axially spaced apart from one another, each of the plurality of strut regions comprise a plurality of struts, each of the struts extending between bridges of circumferentially adjacent bridge regions, and at least when the at least a portion of the sidewall is in a straight configuration, each of the struts extend at a non-perpendicular angle relative to a longitudinal axis of the second elongate tube.

Example 62: A steerable instrument comprising: an elongate member having a proximal portion, a distal portion, an actuation region at the proximal portion, and an articulable region at the distal portion, and: a first elongate tube comprising a sidewall and having a tendon formed of the sidewall, the tendon extending between a proximal end portion at the actuation region and a distal end portion at the articulable region, wherein the tendon is configured to transfer a force applied at the actuation region to the articulable region to cause articulation of the articulable region, and a second elongate tube coaxial with the first elongate tube and comprising a sidewall, wherein at least a portion of the sidewall axially coincides with the articulable region and comprises a plurality of bridge regions and a plurality of struts regions alternating with the plurality of bridge regions along a circumference of the second elongate tube such that the strut regions extend circumferentially between and connect the bridge regions, and wherein: each of the plurality of bridge regions comprise a plurality of bridges axially spaced apart from one another, each of the plurality of strut regions comprise a plurality of struts, each of the struts extending between bridges of circumferentially adjacent bridge regions, and at least when the at least a portion of the sidewall is in a straight configuration, each of the struts extend at a non-perpendicular angle relative to a longitudinal axis of the second elongate tube.

Example 63: The instrument of Example 62, wherein the tendon is circumferentially aligned with one of the bridge regions.

Example 64: The instrument of Example 62 or 63, wherein the first elongate tube comprises a plurality of tendons, and wherein each of the plurality of tendons is circumferentially aligned with a respective one of the bridge regions.

Example 65: The instrument of any one of Examples 62 to 64, wherein the articulable region is configured to bend omnidirectionally.

Example 66: An instrument comprising: an elongate member formed of a tubular sidewall, wherein at least a portion of the tubular sidewall comprises a plurality of bridge regions and a plurality of struts regions alternating with the plurality of bridge regions along a circumference of the elongate member such that the strut regions extend circumferentially between and connect the bridge regions, and wherein: each of the plurality of bridge regions comprise a plurality of bridges axially spaced apart from one another, each of the plurality of strut regions comprise a plurality of struts, each of the struts extending between bridges of circumferentially adjacent bridge regions, and at least when the elongate member is in a straight configuration, within a given strut region, each of the struts extend at an angle relative to an axially adjacent strut.

Example 67: The instrument of Example 66, wherein the at least a portion of the tubular sidewall is configured to bend omnidirectionally.

Example 68: The instrument of Example 66 or 67, wherein the elongate member includes at least two bridge regions and at least two strut regions.

Example 69: The instrument of any one of Examples 66 to 68, wherein the elongate member includes at least four bridge regions and at least four strut regions.

Example 70: The instrument of any one of Examples 66 to 69, wherein the bridges of circumferentially adjacent bridge regions are axially offset from one another.

Example 71: The instrument of any one of Examples 66 to 70, wherein at least some of the bridges surround and define an opening.

Example 72: The instrument of any one of Examples 66 to 71, wherein at least some of the bridges connect to four struts.

Example 73: An instrument comprising: an elongate member formed of a tubular sidewall, wherein at least a portion of the sidewall comprises a plurality of bridge regions and a plurality of struts regions alternating with the plurality of bridge regions along a circumference of the elongate member such that the strut regions extend circumferentially between and connect adjacent bridge regions, and wherein: each of the plurality of bridge regions comprise a plurality of bridges axially spaced apart from one another, each of the plurality of strut regions comprise a plurality of struts, each of the struts extending between bridges of circumferentially adjacent bridge regions, and at least when the at least a portion of the sidewall is in a straight configuration, each of the struts extend along a curved path between bridges.

Example 74: An instrument comprising: an elongate member having a proximal portion, a distal portion, an actuation region at the proximal portion, an articulable region at the distal portion, and: a first elongate tube comprising a first sidewall and having a first flexible region along which the first elongate tube is configured to bend, wherein the first flexible region comprises a plurality of circumferentially extending struts cut from and formed of the first elongate tube, and wherein, at least when the first flexible region is in a straight configuration, each of the struts extend at a non-perpendicular angle relative to a longitudinal axis of the first elongate tube, and a second elongate tube coaxial with the first elongate tube, wherein the second elongate tube comprises a second sidewall having a second flexible region comprising a plurality of helical strands formed of the second sidewall, and wherein the second flexible region is at least partially axially aligned with the first flexible region.

Example 75: The instrument of Example 74, further comprising a third elongate tube coaxial with the first and second elongate tubes.

Example 76: The instrument of Example 74 or 75, wherein the helical strands are first helical strands and the instrument further comprises a third elongate tube comprising a third sidewall having a third flexible region comprising a plurality of second helical strands formed of the third sidewall, and wherein the third flexible region is at least partially axially aligned with the first and second flexible regions.

Example 77: The instrument of Example 76, wherein the first and second helical strands wrap around the circumference of the instrument in opposite circumferential directions.

Example 78: The instrument of any one of Examples 74 to 77, wherein the second elongate tube is shorter than the first elongate tube.

Example 79: The instrument of any one of Examples 74 to 78, wherein a portion of the elongate member axially coinciding with the first and second flexible regions is configured to bend omnidirectionally.

Example 80: The instrument of any one of Examples 74 to 79, wherein axially adjacent struts extend towards opposite axial directions from a shared bridge.

Example 81: The instrument of any one of Examples 74 to 80, wherein the helical strands are first helical strands and the second elongate tube further comprises a third flexible region axially spaced apart from the second flexible region, and wherein the third flexible region comprisess a plurality of second helical strands formed of the second sidewall.

Example 82: The instrument of Example 81, wherein the first and second helical strands wrap around the circumference of the second elongate tube in opposite circumferential directions.

Example 83: The instrument of Example 81, wherein the first and second helical strands wrap around the circumference of the second elongate tube in the same circumferential direction.

Example 84: A steerable instrument comprising: an elongate member having a proximal portion, a distal portion, an actuation region at the proximal portion, and an articulable region at the distal portion, and: a first elongate tube comprising a first sidewall and having a tendon formed of the first sidewall, the tendon extending between a proximal end portion at the actuation region and a distal end portion at the articulable region, wherein the tendon is configured to transfer a force applied at the actuation region to the articulable region to cause articulation of the articulable region, a second elongate tube comprising a second sidewall and having a second flexible region along which the second elongate tube is configured to bend, wherein the second flexible region comprises a plurality of circumferentially extending struts cut from and formed of the second elongate tube, and wherein, at least when the second flexible region is in a straight configuration, each of the struts extend at a non-perpendicular angle relative to a longitudinal axis of the second elongate tube, and a third elongate tube coaxial with the first and second elongate tubes, wherein the third elongate tube comprises a third sidewall having a third flexible region comprising a plurality of helical strands formed of the third sidewall, and wherein the articulable region, second flexible region, and third flexible region are at least partially axially aligned with one another.

Example 85: The instrument of Example 84, wherein the articulable region is configured to bend omnidirectionally.

Example 86: The instrument of Example 84 or 85, wherein the third elongate tube is shorter than the second and first elongate tubes.

Example 87: The instrument of Example any one of Examples 84 to 86, wherein axially adjacent struts extend towards opposite axial directions from a shared bridge.

Example 88: A method of making an instrument, comprising: providing a first elongate tube; cutting a plurality of circumferentially extending struts from a first length of the first elongate tube such that the struts extend at a non-perpendicular angle relative to a longitudinal axis of the first elongate tube; providing a second elongate tube; cutting a plurality of helical strands from a second length of the second elongate tube; and inserting the first elongate tube into a lumen of the second elongate tube, or vice versa, such that the first and second lengths are axially aligned.

Example 89: The method of Example 88, further comprising fixing a portion of the first elongate tube at a distal side of first length to a portion of the second elongate tube at a distal side of the second length.

Example 90: The method of Example 88 or 89, further comprising fixing a portion of the first elongate tube at a proximal side of first length to a portion of the second elongate tube at a proximal side of the second length.

Example 91: The method of Example 88, further comprising: fixing a portion of the first elongate tube at a distal side of first length to a portion of the second elongate tube at a distal side of the second length, and fixing a portion of the first elongate tube at a proximal side of first length to a portion of the second elongate tube at a proximal side of the second length.

Example 92: The method of any one of Examples 88 to 91, further comprising coaxially arranging a third elongate tube relative to the first and second elongate tubes.

Example 93: The method of any one of Examples 88 to 92, further comprising coaxially arranging a fourth elongate tube relative to the first, second, and third elongate tubes.

### CONCLUSION

The cut tube assemblies of the present technology are applicable a variety of medical applications and/or approaches. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-26.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

It will be clear to a person skilled in the art that the scope of the invention is not limited to the examples discussed in the foregoing, but that several amendments and modifications thereof are possible without deviating from the scope of the invention as defined in the attached claims. While the invention has been illustrated and described in detail in the figures and the description, such illustration and description are to be considered illustrative or exemplary only, and not restrictive. The present invention is not limited to the disclosed embodiments but comprises any combination of the disclosed embodiments that can come to an advantage.

Variations to the disclosed embodiments can be understood and effected by a person skilled in the art in practicing the claimed invention, from a study of the figures, the description and the attached claims. In the description and claims, the word “comprising” does not exclude other elements, and the indefinite article “a” or “an” does not exclude a plurality. In fact it is to be construed as meaning “at least one”. The mere fact that certain features are recited in mutually different dependent claims does not indicate that a combination of these features cannot be used to advantage. Any reference signs in the claims should not be construed as limiting the scope of the invention. Features of the above described embodiments and aspects can be combined unless their combining results in evident technical conflicts.

I/We claim:

1. An instrument comprising:
an elongate member having a proximal portion, a distal portion and an intermediate portion extending in a longitudinal direction between the proximal and distal portions, wherein the elongate member further includes:
a first elongate tube comprising a sidewall having a first proximal band, a first distal band, and a first flexible region extending axially between the first proximal and distal bands, wherein the first flexible region comprises a plurality of first strands extending helically around a longitudinal axis of the first elongate tube in a first circumferential direction between the first proximal and distal bands, and
a second elongate tube coaxial with the first elongate tube, wherein the second elongate tube comprises a sidewall having a second proximal band, a second distal band, and a second flexible region extending axially between the second proximal and distal bands, wherein the second flexible region comprises a plurality of second strands extending helically around a longitudinal axis of the second elongate tube in a second circumferential direction between the second proximal and distal bands, the second circumferential direction opposite the first circumferential direction,
wherein the first and second flexible regions are at least partially axially aligned with one another.

2. The instrument of claim 1, wherein the first and second proximal bands are aligned and fixed to one another and the first and second distal bands are aligned and fixed to one another.

3. The instrument of claim 1, wherein the elongate member further comprises a third elongate tube coaxial with the first and second elongate tubes.

4. The instrument of claim 3, wherein the elongate member further comprises a fourth elongate tube coaxial with the first, second, and third elongate tubes.

5. The instrument of claim 1, wherein the first strands extend helically around the first elongate tube at least 360 degrees between the first proximal and distal bands.

6. The instrument of claim 1, wherein the second strands extend around the second elongate tube at least 360 degrees between the second proximal and distal bands.

7. The instrument of claim 1, wherein the first strands extend around 360 degrees of the first elongate tube N times between the first proximal and distal bands, wherein N is an integer of 1 or more.

8. The instrument of claim 1, wherein the second strands extend around 360 degrees of the second elongate tube N times between the second proximal and distal bands, wherein N is an integer of 1 or more.

9. The instrument of claim 1, wherein:
the first helical strands extend around 360 degrees of the first elongate tube N times between the first proximal and distal bands, wherein N is an integer of 1 or more, and
wherein the second helical strands extend around 360 degrees of the second elongate tube N times between the second proximal and distal bands.

10. The instrument of claim 1, wherein:
the first helical strands extend around 360 degrees of the first elongate tube N times between the first proximal and distal bands, wherein N is an integer of 1 or more, and
wherein the second helical strands extend around the second elongate tube more than an integer multiple of 360 degrees between the second proximal and distal bands.

11. An instrument comprising:
an elongate member having a proximal portion, a distal portion, an actuation region at the proximal portion, an articulable region at the distal portion, and:
a first elongate tube comprising a first sidewall and having a first flexible region along which the first elongate tube is configured to bend, and
a second elongate tube coaxial with the first elongate tube, wherein the second elongate tube comprises a second sidewall having a second flexible region comprising a plurality of helical strands formed of the second sidewall, and wherein the second flexible region is at least partially axially aligned with the first flexible region.

12. The instrument of claim 11, wherein the second flexible region is disposed between a first rigid band and a second rigid band, each of the first and second rigid bands formed of the second sidewall.

13. The instrument of claim 12, wherein the strands extend around the second elongate tube at least 360 degrees between the first and second rigid bands.

14. The instrument of claim 12, wherein the strands extend around 360 degrees of the second elongate tube N times between the first and second bands, wherein N is an integer of 1 or more.

15. The instrument of claim 12, wherein the first flexible region is disposed between a first rigid band and a second rigid band, each of the first and second rigid bands formed of the first sidewall.

16. The instrument of claim 15, wherein the first bands of the first and second sidewalls are axially aligned and fixed to one another and the second bands of the first and second sidewalls are axially aligned and fixed to one another.

17. The instrument of claim 11, wherein the elongate member further comprises a third elongate tube coaxial with the first and second elongate tubes.

18. The instrument of claim 17, wherein the elongate member further comprises a fourth elongate tube coaxial with the first, second, and third elongate tubes.

19. An instrument comprising:
an elongate member having a proximal portion, a distal portion, an actuation region at the proximal portion, an articulable region at the distal portion, and:
a first elongate tube comprising a sidewall and having a tendon formed of the sidewall, the tendon extending between the actuation region and the articulable region and configured to transfer a force applied at the actuation region to the articulable region to cause articulation of the articulable region, and wherein the first elongate tube comprises a first flexible region axially coinciding with the articulable region and through which the tendon extends to control articulation, and
a second elongate tube coaxial with the first elongate tube, wherein the second elongate tube comprises a sidewall having a second flexible region comprising a plurality of helical strands formed of the sidewall, and wherein the second flexible region is at least partially axially aligned with the first flexible region.

20. The instrument of claim 19, wherein the first flexible region extends between a first distal band and a first proximal band, each formed of the sidewall of the first elongate tube, and wherein a distal end portion of the tendon is attached to the first distal band.
